(19) United States
(12) Patent Application Publication
(10) Pub. No.: US 2024/0067774 A1
(43) Pub. Date: Feb. 29, 2024

(54) PYRROLE DERIVED COMPOSITIONS AND SYTHESIS METHODS

(71) Applicant: Kennesaw State University Research and Service Foundation, Inc., Kennesaw, GA (US)

(72) Inventors: Graham Collier, Kennesaw, GA (US); Kenneth-John Bell, Powder Springs, GA (US)

(21) Appl. No.: 18/447,804

(22) Filed: Aug. 10, 2023

Related U.S. Application Data
(60) Provisional application No. 63/396,834, filed on Aug. 10, 2022.

(51) Int. Cl. C08G 61/12; H10K 85/10

(52) U.S. Cl. CPC C08G 61/124; H10K 85/113; H10K 85/151; C08G 2261/124; C08G 2261/1412; C08G 2261/148; C08G 2261/18; C08G 2261/228; C08G 2261/312; C08G 2261/324; C08G 2261/3241; C08G 2261/411; H10K 30/30

(57) ABSTRACT

A novel copolymer for use in organic photovoltaics and other related electronic fields and related synthesis pathway. The novel copolymer displays the first example of an H₂DPP co-monomer being directly incorporated into the main chain of a polymer repeat unit to form a co-polymer. An example co-polymer includes H₂DPP-co-ProDOT. The related synthesis pathway displays significant simplicity and eliminates needs for reaction pathways, reagents, conditions, and the like associated with waste, toxicity, and other undesired properties.

### CROSS-REFERENCE TO RELATED APPLICATION

This application is a U.S. non-provisional of and claims the benefit of U.S. Provisional Application No. 63/396,834, filed Aug. 10, 2022, which is hereby incorporated by reference in its entirety.

### TECHNICAL FIELD

This disclosure generally relates to the field of high-performance, conjugated polymers and synthesis processes thereof.

### BACKGROUND

Conjugated polymers are useful materials in various types of organic electronic devices, such as organic photovoltaics (OPVs) (Lu, L, et al., “Recent Advances in Bulk Heterojunction Polymer Solar Cells”, Chem. Rev., 115 (23): 12666-12731 (2015)), organic light-emitting diodes (OLEDs) (Salehi, A., et al., “Recent Advances in OLED Optical Design”, Adv. Funct. Mater., 29 (15): 1808803 (2019)), or electrochromics (Li, X., et al., “Solution-Processable Electrochromic Materials and Devices: Roadblocks and Strategies Towards Large-Scale Applications”, J. Mater. Chem., 7 (41): 12761-12789 (2019)), but one limitation to their utilization in these applications is the difficult and potentially complex nature of their synthesis. Typically, the synthesis of high performance conjugated polymers requires numerous synthetic steps, including air and moisture-free environments or cryogenic conditions, harsh/toxic reagents, and rigorous purifications during the preparation of their monomeric building blocks (Osedach, T. P., et al., “Effect of Synthetic Accessibility on the Commercial Viability of Organic Photovoltaics”, Energy Environ. Sci., 6(3): 711-718 (2013); Po, R., et al., “‘All That Glisters Is Not Gold’: An Analysis of the Synthetic Complexity of Efficient Polymer Donors for Polymer Solar Cells”, Macromolecules, 48 (3): 453-461 (2015); Li, X., et al., “Simplified Synthetic Routes for Low Cost and High Photovoltaic Performance N-Type Organic Semiconductor Acceptors”, Nat. Commun., 10(1): 519 (2019)).

Attempts to simplify synthesis have been made. Po et al. (2015) defined the synthetic complexity of a polymer as being dependent on five parameters, including number of synthetic steps (NSS), the reciprocal yield of monomers (RY), the number of operations required for purification of monomers (NUO), the number of column chromatography purifications (NCC), and the number of hazardous materials used (NHC) (Po, R., et al., “‘All That Glisters Is Not Gold’: An Analysis of the Synthetic Complexity of Efficient Polymer Donors for Polymer Solar Cells”, Macromolecules, 48 (3): 453-461 (2015)). Synthetic simplicity can be comparatively defined where a more synthetically simple polymer will have a lower number associated with it based on Po et al.'s calculations. While this approach has become a useful method for estimating synthetic complexity, McCulloch and coworkers suggest that there are too many inconsistencies in reported synthetic methods, as well as discrepancies in hazard definitions. Thus, they developed the scalability factor (SF), where the NSS and RY of the synthetic route are the main factors influencing these benchmark calculations (Moser, M., et al., “Challenges to the Success of Commercial Organic Photovoltaic Products”, Adv. Energy Mater, 11(18): 2100056 (2021)). Regardless of the approach used to assess the synthetic complexity of conjugated materials, the emphasis on reducing the number of synthetic steps while using starting materials “that could be found in the market in quantities sufficient to produce hundreds of kilograms of polymers” (Min, J., et al., “Evaluation of Electron Donor Materials for Solution-Processed Organic Solar Cells via a Novel Figure of Merit”, Adv. Energy Mater., 7 (18): 1700465 (2017)) provides motivation to explore new structural design motifs with simple chemistries.

Therefore, there exists a need for accessing conjugated polymers suitable for applications ranging from organic photovoltaics to bioelectronics through simple and safe synthetic protocols.

### SUMMARY

It is to be understood that this summary is not an extensive overview of the disclosure. This summary is example and not restrictive and it is intended to neither identify key or critical elements of the disclosure nor delineate the scope thereof. The sole purpose of this summary is to explain and exemplify certain concepts of the disclosure as an introduction to the following complete and extensive detailed description. Citation or identification of any document in this application is not an admission that such document is available as prior art to the present invention.

The present disclosure relates to a polymeric composition for use in organic electronics. The polymeric composition can be used in photovoltaics and electrochromism. The polymeric composition includes but is not limited to a copolymer with a first pyrrolopyrrole-based monomer and a second, electroactive monomer. The first monomer includes pyrrolo[3,2-b]pyrrole (H2DPP). The second monomer includes dioxythiophene-based monomers, such as 3,4-propylenedioxythiophene (ProDOT).

The present disclosure relates to methods including synthetic schemes that comprise low synthetic complexity and reduced toxicities associated with various reagents and pathways.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1 displays representative structures of traditionally studied conjugated polymers and the attributes of utilizing H₂DPP copolymers.

FIG. 2 displays example reaction schemes according to the present disclosure.

FIGS. 3A-3B display example reaction schemes for synthesizing an H₂DPP copolymers.

FIG. 4 displays an example reaction scheme for synthesizing an H₂DPP comonomer.

FIGS. 5A-5B display high-temperature size exclusion chromatography (HT-SEC) measurements of H₂DPP-co-thienopyrroledione (TPD) recovered from Soxhlet thimbles after polymerization in various solvents (FIG. 5A) and UV-vis absorbance spectra of H₂DPP-co-TPD copolymers synthesized in various solvents (FIG. 5B).

FIG. 6 displays an example reaction scheme for synthesis of Br₂DPP via an Fe-catalyzed multi-component reaction using n-decylaniline and a photograph of about 15 grams of the Br₂DPP monomer next to a 20 mL vial to illustrate scalability.

FIG. 7 displays ¹H NMR results of Br₂DPP.

FIG. 8 displays ¹³C NMR results of Br₂DPP.

FIGS. 9A-9B display an example reaction pathway (FIG. 9A) and ¹H NMR spectra of Br₂DPP before (black) and after (red) subjecting the monomer to the reaction conditions (FIG. 9B).

FIG. 10 displays size exclusion chromatography (SEC) elugrams for H₂DPP-co-ProDOT.

FIG. 11 displays ¹H NMR results of H₂DPP-co-ProDOT.

FIGS. 12A-12B display structure and frontier molecular orbital maps of the (ProDOT)₂DPP oligomer used for TD-DFT calculations (FIG. 12A) and normalized ultraviolet-visible (UV-vis) absorbance spectra of the resulting TD-DFT calculations of a (ProDOT)₂DPP oligomer (black), a synthesized (iBuProDOT)₂DecylDPP oligomer in toluene solution (red), and H₂DPP-co-ProDOT copolymer in toluene solution (FIG. 12B).

FIG. 13 displays normalized solution ultraviolet-visible absorbance (black) and fluorescence (red) spectra of H₂DPP-co-ProDOT.

FIGS. 14A-14B display thermal gravimetric analysis of H₂DPP-co-ProDOT temperature ramping (FIG. 14A) and differential scanning calorimetry trace of H₂DPP-co-ProDOT (second heating cycle) cycling (FIG. 14B).

FIGS. 15A-15B display ultraviolet-visible absorbance spectra of H₂DPP-co-ProDOT in solution (black), as a pristine spray-cast film (red), and an electrochemically conditioned film (blue) (FIG. 15A) and cyclic voltammogram traces of H₂DPP-co-ProDOT as a pristine (black) and electrochemically conditioned film (red) (FIG. 15B).

FIGS. 16A-16B display absorbance spectra as a function of applied potential of a H₂DPP-co-ProDOT film spray cast (FIG. 16A) and color coordinates and photographs of a H₂DPP-co-ProDOT film spray cast as a function of applied potential (FIG. 16B).

FIGS. 17A-17B display absorbance as a function of potential (FIG. 17A) and color coordinates of H₂DPP-co-ProDOT films with increasing oxidation potential (FIG. 17B).

FIG. 18 displays contrast retention as a function of number of electrochemical switches for DPP-co-ProDOT films.

FIG. 19 displays an example synthesis of Br₂DPP via Fe-catalyzed multicomponent reaction.

FIG. 20 displays a transetherification reaction for the synthesis of dioctyl-ProDOT.

FIG. 21 displays ¹H NMR results of Dioctyl-ProDOT.

FIG. 22 displays an example synthesis of (ProDOT)₂DPP via direct arylation.

FIG. 23 displays an example synthesis of an H₂DPP-based copolymer.

FIG. 24 displays ¹H NMR results of H₂DPP-co-ProDOT.

### DETAILED DESCRIPTION

The present disclosure can be understood more readily by reference to the following detailed description, examples, drawings, and claims, and their previous and following description. However, before the present compositions, systems, and/or methods are disclosed and described, it is to be understood that this disclosure is not limited to the specific devices, systems, and/or methods disclosed unless otherwise specified, as such can, of course, vary. It is also to be understood that the terminology used herein is for the purpose of describing particular aspects only and is not intended to be limiting.

### I. Definitions

It should be appreciated that this disclosure is not limited to the compositions and methods described herein. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

Unless defined otherwise, all composition percentage values used herein are given in terms of weight percentage.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or example language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, the term “alkyl” refers to an unsubstituted or substituted linear or branched alkyl group containing the indicated number of carbon atoms. If no number is indicated, then alkyl (optionally including any substituents on alkyl) may contain 1 to 16 carbon atoms. Preferably, the alkyl group contains 1 to 10 carbon atoms, alternatively 1 to 7 carbon atoms, or alternatively 1 to 4 carbon atoms. Examples of alkyl include methyl, ethyl, propyl, isopropyl, butyl, iso-, sec- and tert-butyl, pentyl, hexyl, heptyl, 3-ethylbutyl, and the like. Examples of substituents on alkyl include 1, 2, or 3 groups independently selected from hydroxy, amino, amido, oxa, carboxy, alkyl carboxy, carbonyl, alkoxy, amido, carbamate, carbonate, halogen, phenyl, benzyl, thiol, and combinations thereof. “Alkylene” means a divalent alkyl group, such as —CH2-, —CH2CH2-, —CH2CH2CH2-, —CH2CH(CH3)CH2-, and —CH2CH2CH2CH2-.

“Haloalkyl” refers to an alkyl group as defined above substituted with one or more halogen atoms, where each halogen is independently F, Cl, Br or I. A preferred halogen is F. Preferred haloalkyl groups contain 1-6 carbons, more preferably 1-4 carbons, and still more preferably 1-2 carbons. “Haloalkyl” includes perhaloalkyl groups, such as —CF3- or —CF2CF3-. “Haloalkylene” means a divalent haloalkyl group, such as —CH2CF2-.

The terms “halogen” or “halo” indicate fluorine, chlorine, bromine, and iodine.

“Cycloalkyl” refers to an unsubstituted or substituted cyclic hydrocarbon containing the indicated number of ring carbon atoms. If no number is indicated, then cycloalkyl may contain 3 to 12 ring carbon atoms. Preferred are C3-C8 cycloalkyl groups, C3-C7 cycloalkyl, more preferably C4-C7 cycloalkyl, and still more preferably C5-C6 cycloalkyl. Examples of cycloalkyl include cyclopropyl, cyclobutyl, cyclopentyl, cyclohexyl, cycloheptyl and cyclooctyl. Examples of substituents on cycloalkyl include 1, 2, or 3 groups independently selected from alkyl, hydroxy, amino, amido, oxa, carbonyl, alkoxy, amido, carbamate, carbonate, halo, phenyl, benzyl, and combinations thereof. “Cycloalkylene” means a divalent cycloalkyl group, such as 1,2-cyclohexylene, 1,3-cyclohexylene, or 1,4-cyclohexylene.

“Heterocycloalkyl” refers to a cycloalkyl ring or ring system as defined above in which at least one ring carbon has been replaced with a heteroatom selected from nitrogen, oxygen, and sulfur. The heterocycloalkyl ring is optionally fused to or otherwise attached to other heterocycloalkyl rings and/or non-aromatic hydrocarbon rings and/or phenyl rings. Preferred heterocycloalkyl groups have from 5 to 7 members. More preferred heterocycloalkyl groups have 5 or 6 members. Heterocycloalkylene means a divalent heterocycloalkyl group.

“Aryl” refers to an unsubstituted or substituted aromatic hydrocarbon ring system containing at least one aromatic ring. The aryl group contains the indicated number of ring carbon atoms. If no number is indicated, then aryl may contain 6 to 14 ring carbon atoms. The aromatic ring may optionally be fused or otherwise attached to other aromatic hydrocarbon rings or non-aromatic hydrocarbon rings. Examples of aryl groups include phenyl, naphthyl, and biphenyl. Preferred examples of aryl groups include phenyl. Examples of substituents on aryl include 1, 2, or 3 groups independently selected from alkyl, hydroxy, amino, amido, oxa, carboxy, alkyl carboxy, carbonyl, alkoxy, amido, carbamate, carbonate, halo, phenyl, benzyl, and combinations thereof. “Arylene” means a divalent aryl group, for example 1,2-phenylene, 1,3-phenylene, or 1,4-phenylene.

“Heteroaryl” refers to an aryl ring or ring system, as defined above, in which at least one ring carbon atom has been replaced with a heteroatom selected from nitrogen, oxygen, and sulfur. The heteroaryl ring may be fused or otherwise attached to one or more heteroaryl rings, aromatic or nonaromatic hydrocarbon rings or heterocycloalkyl rings. Examples of heteroaryl groups include pyridyl, furyl, and thienyl. “Heteroarylene” means a divalent heteroaryl group.

“Alkoxy” refers to an alkyl group attached to the parent molecular moiety through an oxygen bridge. Examples of alkoxy groups include, for instance, methoxy, ethoxy, propoxy and isopropoxy. “Aryloxy” refers to an aryl group attached to a parent molecular moiety through an oxygen bridge. Examples include phenoxy. “Cyclic alkoxy” means a cycloalkyl group attached to the parent moiety through an oxygen bridge.

“Alkylamine” refers to an alkyl group attached to the parent molecular moiety through an —NH bridge. Alkyleneamine means a divalent alkylamine group, such as —CH2CH2NH—.

“Ester” refers to a class of organic compounds having the general formula RCOOR′, wherein R and R′ are any organic combining groups. R and R′ may be selected from functional groups comprising alkyls, substituted alkyls, alkylene, haloalkyls, cycloalkyls, heterocyloalkyls, aryls, heteroaryls, alkoxys, cycloalkoxys, alkylamines, siloxanyls, silyls, alkyleneoxys, oxaalkylenes, and the like. Definitions for the above mentioned functional groups are provided herein.

As used herein, “esterification” refers to a reaction producing an ester. The reaction often involves an alcohol and a Bronsted acid (such as a carboxylic acid, sulfuric acid, or phosphoric acid). Furthermore, the term “transesterification” refers to the reaction of an alcohol molecule and a pre-existing ester molecule react to form a new ester. In some aspects, transesterification can be mediated by other compounds, such as carbonyldiimidazole.

“Siloxanyl” refers to a structure having at least one Si—O—Si bond. Thus, for example, siloxanyl group means a group having at least one Si—O—Si group (i.e. a siloxane group), and siloxanyl compound means a compound having at least one Si—O—Si group. “Siloxanyl” encompasses monomeric (e.g., Si—O—Si) as well as oligomeric/polymeric structures (e.g., [Si—O]n-, where n is 2 or more). Each silicon atom in the siloxanyl group is substituted with independently selected RA groups (where RA is as defined in formula A options (b)-(i)) to complete their valence.

“Silyl” refers to a structure of formula R3Si— and “siloxy” refers to a structure of formula R3Si—O—, where each R in silyl or siloxy is independently selected from trimethylsiloxy, C1-C8 alkyl (preferably C1-C3 alkyl, more preferably ethyl or methyl), and C3-C8 cycloalkyl.

“Alkyleneoxy” refers to groups of the general formula -(alkylene-O)p- or —(O-alkylene)p-, wherein alkylene is as defined above, and p is from 1 to 200, or from 1 to 100, or from 1 to 50, or from 1 to 25, or from 1 to 20, or from 1 to 10, wherein each alkylene is independently optionally substituted with one or more groups independently selected from hydroxyl, halo (e.g., fluoro), amino, amido, ether, carbonyl, carboxyl, and combinations thereof. If p is greater than 1, then each alkylene may be the same or different and the alkyleneoxy may be in block or random configuration. When alkyleneoxy forms a terminal group in a molecule, the terminal end of the alkyleneoxy may, for instance, be a hydroxy or alkoxy (e.g., HO—[CH2CH2O]p- or CH3O—[CH2CH2O]p-). Examples of alkyleneoxy include polymethyleneoxy, polyethyleneoxy, polypropyleneoxy, polybutyleneoxy, and poly(ethyleneoxy-co-propyleneoxy).

“Oxaalkylene” refers to an alkylene group as defined above where one or more non-adjacent CH2 groups have been substituted with an oxygen atom, such as —CH2CH2OCH(CH3)CH2-. “Thiaalkylene” refers to an alkylene group as defined above where one or more non-adjacent CH2 groups have been substituted with a sulfur atom, such as CH2CH2SCH(CH3)CH2-.

The term “linking group” refers to a moiety that links the polymerizable group to the parent molecule. The linking group may be any moiety that does not undesirably interfere with the polymerization of the compound of which it is a part. For instance, the linking group may be a bond, or it may comprise one or more alkylene, haloalkylene, amide, amine, alkyleneamine, carbamate, carboxylate (—CO2-), disulfide, arylene, heteroarylene, cycloalkylene, heterocycloalkylene, alkyleneoxy, oxaalkylene, thiaalkylene, haloalkyleneoxy (alkyleneoxy substituted with one or more halo groups, e.g., —OCF2-, —CF2CF2-, —CF2CH2-), siloxanyl, alkylenesiloxanyl, thiol, or combinations thereof. The linking group may optionally be substituted with 1 or more substituent groups. Suitable substituent groups may include those independently selected from alkyl, halo (e.g., fluoro), hydroxyl, HO-alkyleneoxy, CH3O-alkyleneoxy, siloxanyl, siloxy, siloxy-alkyleneoxy-, siloxy-alkylene-alkyleneoxy- (where more than one alkyleneoxy groups may be present and wherein each methylene in alkylene and alkyleneoxy is independently optionally substituted with hydroxyl), ether, amine, carbonyl, carbamate, and combinations thereof. The linking group may also be substituted with a polymerizable group, such as (meth)acrylate (in addition to the polymerizable group to which the linking group is linked).

Preferred linking groups include C1-C8 alkylene (preferably C2-C6 alkylene) and C1-C8 oxaalkylene (preferably C2-C6 oxaalkylene), each of which is optionally substituted with 1 or 2 groups independently selected from hydroxyl and siloxy. Preferred linking groups also include carboxylate, amide, C1-C8 alkylene-carboxylate-C1-C8 alkylene, or C1-C8 alkylene-amide-C1-C8 alkylene.

As used herein, “pyrrole” refers to a heterocyclic compound (e.g. C₄H₅N) that has a ring comprising four carbon atoms and one nitrogen atom, polymerizes readily in air, and is the parent compound of many biologically important substances (such as bile pigments, porphyrins, and chlorophyll). The term pyrrole used herein refers to pyrrole (C₅H₅N), derivatives of pyrrole (e.g., indole), substituted pyrroles, as well as metal pyrrolide compounds.

When the linking group is comprised of combinations of moieties as described above (e.g., alkylene and cycloalkylene), the moieties may be present in any order. For instance, if in Formula E below, L is indicated as being -alkylene-cycloalkylene-, then Rg-L may be either Rg-alkylene-cycloalkylene-, or Rg-cycloalkylene-alkylene-. Notwithstanding this, the listing order represents the preferred order in which the moieties appear in the compound starting from the terminal polymerizable group (Rg) to which the linking group is attached. For example, if in Formula E, L and L2 are indicated as both being alkylene-cycloalkylene, then Rg-L is preferably Rg-alkylene-cycloalkylene- and -L2-Rg is preferably -cycloalkylene-alkylene-Rg.

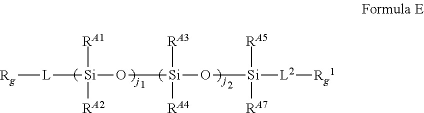

As used herein, a “coupling reaction” is a reaction or reaction sequence in which the net reaction is the connection of carbon skeletons of two compounds containing a common functional group.

As used herein, “oxidation” refers to a chemical process by which an atom of an element gains bonds to more electronegative elements, most commonly oxygen. In this process, the oxidized element increases its oxidation state, which represents the charge of an atom. Oxidation reactions are commonly coupled with “reduction” reactions, wherein the oxidation state of the reduced atom decreases.

As used herein, a “redox reaction” or “reduction oxidation reaction” refers to a type of chemical reaction that involves a transfer of electrons between two species. An oxidation-reduction reaction is any chemical reaction in which the oxidation number of a molecule, atom, or ion changes by gaining or losing an electron.

As used herein, the term “in air” refers to a set of reaction conditions. Such conditions may comprise ambient atmosphere conditions not needing inert gasses to run a reaction. This is advantageous because it eliminates the need for specialty glassware or purchasing nitrogen or argon gas cylinders.

As used herein, the “visible spectrum” refers to a range of wavelengths within the electromagnetic spectrum, wherein the range spans about 380 nm to 700 nm. The visible spectrum may be broken up into different wavelength regions corresponding to colors including red, orange, yellow, green, blue, indigo, and violet. Certain ranges of wavelengths falling in the visible spectrum have been known to cause damage to human eyes.

As used herein, the “ultraviolet (UV) spectrum” refers to a range of wavelengths within the electromagnetic spectrum, wherein the range spans about 10 nm to 400 nm.

As used herein, “light absorption” is defined as the phenomenon wherein electrons absorb the energy of incoming light waves (i.e. photons) and change their energy state. In order for this to occur, the incoming light waves must be at or near the energy levels of the electrons. The resultant absorption patterns characteristic to a given material may be displayed using an “absorption spectrum”, wherein an “absorption spectrum” shows the change in absorbance of a sample as a function of the wavelength of incident light and may be measured using a spectrophotometer. Unique to an “absorption spectrum” is an “absorption peak”, wherein the frequency or wavelength of a given sample exhibits the maximum or the highest spectral value of light absorption. With regards to light absorption of wavelengths of light corresponding to the visible spectrum, a material or matter absorbing light waves of certain wavelengths of the visible spectrum may cause an observer to not see these wavelengths in the reflected light.

As used herein, “fluorescence” refers to a type of luminescence that occurs in gas, liquid or solid matter. Fluorescence occurs following the absorption of light waves (i.e. photons), which may promote an electron from the ground state promoted to an excited state. In fluorescence, the spin of the electron is still paired with the ground state electron, unlike phosphorescence. As the excited electron returns to the ground state, it emits a photon of lower energy, corresponding to a longer wavelength, than that of the absorbed photon.

As used herein, “phosphorescence” refers to a phenomenon of delayed luminescence that corresponds to the radiative decay of an excited electron from the molecular triplet state. As a general property, phosphorescence represents a challenge of chemical physics due to the spin prohibition of the underlying triplet-singlet photon emission and because its analysis embraces a deep knowledge of electronic molecular structure. Phosphorescence is the simplest physical process which provides an example of spin-forbidden transformation with a characteristic spin selectivity and magnetic field dependence, being the model also for more complicated chemical reactions and for spin catalysis applications. Phosphorescence is commonly viewed as the alternative method of photon emission with regards to fluorescence. Methods exist in the art to increase the amount of fluorescence versus phosphorescence emission, such as the use of heavy metals to increase spin coupling.

As used herein, the “quantum yield (Φ)” is a measure of the efficiency of photon emission as defined by the ratio of the number of photons emitted to the number of photons absorbed.

As used herein, “anisotropic” describes a material wherein a given property of said material depends on the direction in which it is measured. Moreover, something that is “anisotropic” changes in size or in its physical properties according to the direction in which it is measured. Examples of anisotropic materials may comprise graphite, carbon fiber, nanoparticles, etc.

As used herein, “isotropic” describes a material wherein a given property of said material does not depend on the direction in which it is measured. Moreover, something that is “isotropic” remains constant in size or in its physical properties according to the direction in which it is measured.

As used herein, “ligand” refers to an ion or neutral molecule that bonds to a central metal atom or ion. Example ligands may comprise PVP, PVA, DSDMA, and other molecules capable of bonding to a central metal atom. Metal atoms may comprise a variety of metals including, but not limited to, noble metals such as gold. Ligands have at least one donor with an electron pair used to form covalent bonds with the metal central atom.

As used herein, an “intermediate ligand” refers to a ligand temporarily conjugated to a metal atom that is further exchanged to allow the conjugation of an alternate ligand.

As used herein, the terms “physical absorption” or “chemical absorption” refer to processes in which atoms, molecules, or particles enter the bulk phase of a gas, liquid, or solid material and are taken up within the volume. Absorption in this manner may be driven by solubility, concentration gradients, temperature, pressure, and other driving forces known in the art.

As used herein, “adsorption” is defined as the deposition of a species onto a surface. The species that gets adsorbed on a surface is known as an adsorbate, and the surface on which adsorption occurs is known as an adsorbent. Examples of adsorbents may comprise clay, silica gel, colloids, metals, nanoparticles etc. Adsorption may occur via chemical or physical adsorption. Chemical adsorption may occur when an adsorbate is held to an adsorbent via chemical bonds, whereas physical adsorption may occur when an adsorbate is joined to an adsorbent via weak van der Waal's forces.

As used herein, “colloid” refers to dispersions of wherein one substance is suspended in another. Many examples of colloids in the art contain polymers. In this aspect, polymers may be adsorbed or chemically attached to the surface of particles suspended in the colloid, or the polymers may freely move in the colloidal suspension. The presence of polymers on particles in the suspension may directly relate to “colloidal stability,” wherein “colloidal stability” refers to the tendency of a colloidal suspension to undergo sedimentation. Sedementation would result in the falling of particles out of a colloid. Polymers adsorbed or chemically attached to a particle may affect its colloidal stability.

As used herein, the term “diffusion” refers to the process wherein there is a net flow of matter from one region to another. An example of such process is “surface diffusion,” wherein particles move from one area of the surface of a subject to another area of the same surface. This can be caused by thermal stress or applied pressure.

As used herein, a “surfactant” refers to a substance that, when added to a liquid, reduces its surface tension, thereby increasing its spreading and wetting properties. Typical surfactants may be partly hydrophilic and partly lipophilic.

As used herein, the term “wetting agent” refers to a specific class of surfactant, wherein a wetting agent reduces the surface tension of water and thus allows a liquid to more easy spread on or “wet” a surface. The high surface tension of water causes problems in many industrial processes where water-based solutions are used, as the solution is not able to wet the surface it is applied to. Wetting agents are commonly used to reduce the surface tension of water and thus help the water-based solutions to spread.

“Target macromolecule” means the macromolecule being synthesized from the reactive monomer mixture comprising monomers, macromers, prepolymers, cross-linkers, initiators, additives, diluents, and the like.

The term “polymerizable compound” means a compound containing one or more polymerizable groups. The term encompasses, for instance, monomers, macromers, oligomers, prepolymers, cross-linkers, and the like.

“Polymerizable groups” are groups that can undergo chain growth polymerization, such as free radical and/or cationic polymerization, for example a carbon-carbon double bond which can polymerize when subjected to radical polymerization initiation conditions. Non-limiting examples of free radical polymerizable groups include (meth)acrylates, styrenes, vinyl ethers, (meth)acrylamides, N-vinyllactams, N-vinylamides, O-vinylcarbamates, O-vinylcarbonates, and other vinyl groups. Preferably, the free radical polymerizable groups comprise (meth)acrylate, (meth)acrylamide, N-vinyl lactam, N-vinylamide, and styryl functional groups, and mixtures of any of the foregoing. More preferably, the free radical polymerizable groups comprise (meth)acrylates, (meth)acrylamides, and mixtures thereof. The polymerizable group may be unsubstituted or substituted. For instance, the nitrogen atom in (meth)acrylamide may be bonded to a hydrogen, or the hydrogen may be replaced with alkyl or cycloalkyl (which themselves may be further substituted).

Any type of free radical polymerization may be used including but not limited to bulk, solution, suspension, and emulsion as well as any of the controlled radical polymerization methods such as stable free radical polymerization, nitroxide-mediated living polymerization, atom transfer radical polymerization, reversible addition fragmentation chain transfer polymerization, organotellurium mediated living radical polymerization, and the like.

A “monomer” is a mono-functional molecule which can undergo chain growth polymerization, and in particular, free radical polymerization, thereby creating a repeating unit in the chemical structure of the target macromolecule. Some monomers have di-functional impurities that can act as cross-linking agents. A “hydrophilic monomer” is also a monomer which yields a clear single phase solution when mixed with deionized water at 25° C. at a concentration of 5 weight percent. A “hydrophilic component” is a monomer, macromer, prepolymer, initiator, cross-linker, additive, or polymer which yields a clear single phase solution when mixed with deionized water at 25° C. at a concentration of 5 weight percent. A “hydrophobic component” is a monomer, macromer, prepolymer, initiator, cross-linker, additive, or polymer which is slightly soluble or insoluble in deionized water at 25° C.

A “macromolecule” is an organic compound having a number average molecular weight of greater than 1500, and may be reactive or non-reactive.

A “macromonomer” or “macromer” is a macromolecule that has one group that can undergo chain growth polymerization, and in particular, free radical polymerization, thereby creating a repeating unit in the chemical structure of the target macromolecule. Typically, the chemical structure of the macromer is different than the chemical structure of the target macromolecule, that is, the repeating unit of the macromer's pendent group is different than the repeating unit of the target macromolecule or its mainchain. The difference between a monomer and a macromer is merely one of chemical structure, molecular weight, and molecular weight distribution of the pendent group. As a result, and as used herein, the patent literature occasionally defines monomers as polymerizable compounds having relatively low molecular weights of about 1,500 Daltons or less, which inherently includes some macromers. In particular, monomethacryloxypropyl terminated mono-n-butyl terminated polydimethylsiloxane (molecular weight=500-1500 g/mol) (mPDMS) and mono-(2-hydroxy-3-methacryloxypropyl)-propyl ether terminated mono-n-butyl terminated polydimethylsiloxane (molecular weight=500-1500 g/mol) (OH-mPDMS) may be referred to as monomers or macromers. Furthermore, the patent literature occasionally defines macromers as having one or more polymerizable groups, essentially broadening the common definition of macromer to include prepolymers. As a result, and as used herein, di-functional and multi-functional macromers, prepolymers, and crosslinkers may be used interchangeably.

A “polymer” is a target macromolecule composed of the repeating units of the monomers used during polymerization. Example polymers may comprise poly(ethylene glycol) (PEG), polycarbonate, poly(vinyl alcohol) (PVA), polyvinylpyrrolidone (PVP), polystyrene (PS), polycaprolactone (PCL), ethylene oligomers or polyethylene (PE), polypropylene (PP), poly(methyl methacrylate) (PMMA), and other polymers known in the art.

As used herein, the term “thermoplastic” refers to a property of polymers, wherein the polymer may be melted, solidified, and then successfully melted and solidified again. This process may be repeated several times for thermoplastic polymers without loss of functionality.

As used herein, the term “thermoset” refers to a property of polymers, wherein a thermoset polymer forms well-defined, irreversible, chemical networks that tend to grow in three dimensional directions through the process of curing, which can either occur due to heating or through the addition of a curing agent, therefore causing a crosslinking formation between its chemical components, and giving the thermoset a strong and rigid structure that can be added to other materials to increase strength. Once a thermoset polymer has formed networks during curing, the polymer cannot be re-cured to set in a different manner.

A “homopolymer” is a polymer made from one monomer; a “copolymer” is a polymer made from two or more monomers; a “terpolymer” is a polymer made from three monomers. A “block copolymer” is composed of compositionally different blocks or segments. Diblock copolymers have two blocks. Triblock copolymers have three blocks. “Comb or graft copolymers” are made from at least one macromer.

A “repeating unit” is the smallest group of atoms in a polymer that corresponds to the polymerization of a specific monomer or macromer.

An “initiator” is a molecule that can decompose into radicals which can subsequently react with a monomer to initiate a free radical polymerization reaction. A thermal initiator decomposes at a certain rate depending on the temperature; typical examples are azo compounds such as 1,1′-azobisisobutyronitrile and 4,4′-azobis(4-cyanovaleric acid), peroxides such as benzoyl peroxide, tert-butyl peroxide, tert-butyl hydroperoxide, tert-butyl peroxybenzoate, dicumyl peroxide, and lauroyl peroxide, peracids such as peracetic acid and potassium persulfate as well as various redox systems. A photo-initiator decomposes by a photochemical process; typical examples are derivatives of benzil, benzoin, acetophenone, benzophenone, camphorquinone, and mixtures thereof as well as various monoacyl and bisacyl phosphine oxides and combinations thereof.

A “cross-linking agent” is a di-functional or multi-functional monomer or macromer which can undergo free radical polymerization at two or more locations on the molecule, thereby creating branch points and a polymeric network. Common examples are bis(2-methacryloyl)oxyethyl disulfide (DSDMA), cetyltrimethylammonium bromide (CTAB), cetyltrimethylammonium chloride (CTAC), ethylene glycol dimethacrylate, tetraethylene glycol dimethacrylate, trimethylolpropane trimethacrylate, methylene bisacrylamide, triallyl cyanurate, and the like.

A “prepolymer” is a reaction product of monomers which contains remaining polymerizable groups capable of undergoing further reaction to form a polymer.

A “polymeric network” is a cross-linked macromolecule that can swell but cannot dissolve in solvents. “Hydrogels” are polymeric networks that swell in water or aqueous solutions, typically absorbing at least 10 weight percent water. “Silicone hydrogels” are hydrogels that are made from at least one silicone-containing component with at least one hydrophilic component. Hydrophilic components may also include non-reactive polymers.

An “interpenetrating polymeric network” comprises two or more networks which are at least partially interlaced on the molecular scale but not covalently bonded to each other and which cannot be separated without braking chemical bonds. A “semi-interpenetrating polymeric network” comprises one or more networks and one or more polymers characterized by some mixing on the molecular level between at least one network and at least one polymer. A mixture of different polymers is a “polymer blend.” A semi-interpenetrating network is technically a polymer blend, but in some cases, the polymers are so entangled that they cannot be readily removed.

The terms “reactive mixture” and “reactive monomer mixture” refer to the mixture of components (both reactive and non-reactive) which are mixed together and when subjected to polymerization conditions form the conventional or silicone hydrogels of the present invention as well as contact lenses made therefrom. The reactive monomer mixture may comprise reactive components such as the monomers, macromers, prepolymers, cross-linkers, and initiators, additives such as wetting agents, release agents, polymers, dyes, light absorbing compounds such as UV absorbers, pigments, dyes and photochromic compounds, any of which may be reactive or non-reactive but are capable of being retained within the resulting biomedical device, as well as pharmaceutical and nutraceutical compounds, and any diluents. It will be appreciated that a wide range of additives may be added based upon the biomedical device which is made and its intended use. Concentrations of components of the reactive mixture are expressed as weight percentages of all components in the reactive mixture, excluding diluent. When diluents are used, their concentrations are expressed as weight percentages based upon the amount of all components in the reactive mixture and the diluent.

“Reactive components” are the components in the reactive mixture which become part of the chemical structure of the polymeric network of the resulting hydrogel by covalent bonding, hydrogen bonding, electrostatic interactions, the formation of interpenetrating polymeric networks, or any other means.

The term “multi-functional” refers to a component having two or more polymerizable groups. The term “mono-functional” refers to a component having one polymerizable group.

As used herein, “electrochromism” refers to a phenomenon exhibited by certain electroactive materials with reversible and significant visible change in absorbance spectra. Electrochromism further refers to a reversible change in optical properties of a material caused by redox reactions. Redox reactions can be initiated when a material is placed on the surface of an electrode. When an electrochromic material is capable of showing several colors, it is known as “polyelectrochromic”. Changes in color may occur when a chromophore is forced to change its absorption spectrum by the application of electric potential. As a non-limiting example, the absorption may change from the UV region into the visible region.

As used herein, “organic photovoltaics (OPV)” refers to a field focusing on creating devices that convert solar energy to electrical energy. An OPV device may include one or several photoactive materials sandwiched between electrodes.

As used herein, “organic light emitting diodes (OLEDs)” refer to solid-state light devices that use flat light emitting technology in addition to two conductors between which a series of organic thin films are kept.

As used herein, “pi electrons” or “π electrons” refer to electrons residing within certain molecular orbitals. As non-limiting examples, pi electrons may reside in the pi bonds of a double bond, a triple bond, and a conjugated p orbital. As a non-limiting example, the allyl carbanion has four pi electrons.

As used herein, “antibonding pi electrons” or “π* electrons” refer to electrons residing within certain molecular orbitals. As a non-limiting example, antibonding pi electrons reside in antibonding molecular orbitals of pi bonds where antibonding orbitals weaken chemical bonds and raise energies associated therewith. As a non-limiting example, ethylene comprises a π* molecular orbital with four orbital lobes.

As used herein, “frontier molecular orbital theory (FMOT)” refers to the idea of frontier molecular orbitals of a compound at the frontier of electron occupation. This relates to a highest-occupied molecular orbital (HOMO) and a lowest-unoccupied molecular orbital (LUMO).

As used herein, “torsional strain” refers to destabilization of a molecule caused by the eclipsing of groups present on the adjacent atoms. It is the difference in the energy between a staggered and eclipsed conformation and can be qualified using a Newman projection.

Unless otherwise indicated, ratios, percentages, parts, and the like are by weight.

Unless otherwise indicated, numeric ranges, for instance as in “from 2 to 10,” are inclusive of the numbers defining the range (e.g., 2 and 10).

### II. Polymer Compositions

The present disclosure relates to polymers. Polymers can be used in a variety of electronics. Polymers can be synthesized in a variety of structures depending on a desired application. As non-limiting examples, polymers can be structured as thin sheets, thin films, sheets, films, nanoparticles, macroparticles, coatings, and the like. Polymers include but are not limited to high-performance conjugated polymers. Polymers include copolymers between a pyrrolopyrrole-based monomer and an additional polymeric repeating unit. As a non-limiting example, a pyrrolopyrrole-based monomer may include pyrrolo[3,2-b]pyrrole (H₂DPP). As a non-limiting example, an additional polymeric repeating unit may be 3,4-propylenedioxythiophene (ProDOT), though other such repeating units are possible.

The present disclosure relates to synthesis of a variety of polymers. Polymers may include many functional groups disclosed herein. As a non-limiting example, polymers incorporating pyrollo-functional groups may be of interest. H₂DPP is a building block that meets criteria described above for simplifying synthesis. H₂DPP additionally represents a highly-tailorable chromophore. This chromophore possesses properties amendable to technologically relevant applications (e.g. organic photovoltaics (OPVs), organic light emitting diodes (OLEDs), and other such applications). H₂DPPs represent a class of electron rich, 10π-electron chromophores that are easily synthesized in a single step and highly tailorable through simple structural modification. H₂DPPs are capable of being quickly formed by reactions between aldehydes, anilines, and butanedione in the presence of acetic acid (Krzeszewski, M., et al., “The Tetraarylpyrrolo[3,2-b]Pyrroles—From Serendipitous Discovery to Promising Heterocyclic Optoelectronic Materials”, Acc. Chem. Res., 50 (9): 2334-2345 (2017)). Synthesis of H₂DPPs can be performed in air and does not require column chromatography for purification (Janiga, A., et al., “Synthesis and Optical Properties of Tetraaryl-1,4-Dihydropyrrolo[3,2-b]Pyrroles”, Asian J. Org. Chem, 2(5): 411-415 (2013)). Chromophores may be considered tunable where functional groups at their peripheries are easily manipulated. Manipulation may include but is not limited to replacement, removal, cross-linking, and the like. Functionalization and tunability may be related to starting groups.

As a non-limiting example, acetaminophen may be used as a starting group. Acetaminophen may be used as a starting material for introducing solubilizing handles to H₂DPP monomers. Kirkus, M., et al., “Synthesis and Optical Properties of Pyrrolo[3,2-b]pyrrole-2,5(1H,4H)-dione (iDPP)-Based Molecules,” J. Phys. Chem. A, 117(13): 2782-2789 (2013); Cho, H.H., et al., “Synthesis and Side-Chain Engineering of Phenylnaphthalenediimide (PNDI)-Based n-type Polymers for Efficient All-Polymer Solar Cells,” J. Mater. Chem. A, 5(11): 5449-5459 (2017). As shown in the scheme below, acetaminophen enables a more robust synthetic pathway for side chain engineering that exploits a two-step process starting with the alkylation of acetaminophen followed by deprotection via hydrolysis of the acetyl group.

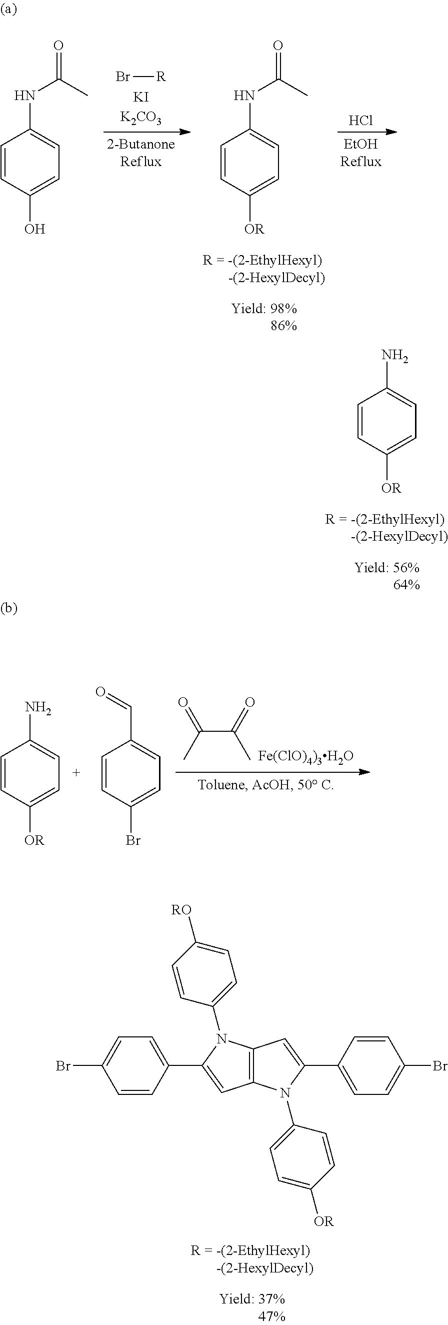

The present disclosure relates to optimized reaction conditions and expanded functional group tolerance. This allows families of chromophores with diverse functionality to be readily synthesized (Krzeszew ski, M., et al., “Tetraaryl-, Pentaaryl-, and Hexaaryl-1,4-Dihydropyrrolo[3,2-b]Pyrroles: Synthesis and Optical Properties”, J. Org. Chem., 79 (7): 3119-3128 (2014); Tasior, M., et al., “Fe(III)-Catalyzed Synthesis of Pyrrolo[3,2-b]Pyrroles: Formation of New Dyes and Photophysical Studies”, Org. Chem. Front., 6 (16): 2939-2948 (2019); Tasior, M., et al., “Method for the Large-Scale Synthesis of Multifunctional 1,4-Dihydro-Pyrrolo[3,2-b]Pyrroles”, J. Org. Chem., 85 (21): 13529-13543 (2020)). The variety of functional groups capable of being incorporated into the periphery of H₂DPPs allows chromophores to exhibit tunable optical properties (Tasior, M., et al., “Synthesis of Bis(Arylethynyl)Pyrrolo[3,2-b]Pyrroles and Effect of Intramolecular Charge Transfer on Their Photophysical Behavior”, Chem. Euro. J., 25 (2): 598-608 (2019); Banasiewicz, M., et al., “Electronic Communication in Pyrrolo[3,2-b]Pyrroles Possessing Sterically Hindered Aromatic Substituents”, Eur. J. Org. Chem., 2019 (31-32): 5247-5253 (2019)), aggregation-induced emission (Sadowski, B., et al., “Tetraphenylethylenepyrrolo[3,2-b]Pyrrole Hybrids as Solid-State Emitters: The Role of Substitution Pattern”, Org. Lett., 20 (11): 3183-3186 (2018); Hatanaka, S., et al., “Tris(Pentafluorophenyl)Borane-Pyrrolo[3,2-b]Pyrrole Hybrids: Solid-State Structure and Crystallization-Induced Enhanced Emission”, ChemPhotoChem, 4 (2): 138-143 (2020)), and two-photon fluorescence (Qin, Y., et al., “Direct Observation of Different One- and Two-Photon Fluorescent States in a Pyrrolo[3,2-b]Pyrrole Fluorophore”, J. Phys. Chem. Lett., 11 (12): 4866-4872 (2020)). Such tunability can also be impacted by cross couplings that systematically alter the resulting properties of a chromophore. Ease of tunability and versatility allows the use of H₂DPPs in various optoelectronic applications (e.g. OPVs, OLEDs, etc.).

As it relates to photovoltaic studies, a small-molecule acceptor-donor-acceptor system via functionalization of the 2- and 5- position aryl rings of H₂DPP with dicyanovinylenes has been shown (Domínguez, R., et al., “Pyrrolo[3,2-b]Pyrrole as the Central Core of the Electron Donor for Solution-Processed Organic Solar Cells”, Chempluschem. 82 (7): 1096-1104 (2017)). This chromophore was used as the light-harvesting material in OPV devices and achieved a maximum PCE of 1.06%. Other efforts have involved extending the conjugated pathway between the H₂DPP core and acceptor region through the addition of thienyl or carbazole moieties, which resulted in a PCE of up to 6.56% when using these dyes in a dye-sensitized solar cell (DSSC) (Wang, J., et al., “Organic Dyes Based on Tetraaryl-1,4-Dihydropyrrolo-[3,2-b]Pyrroles for Photovoltaic and Photocatalysis Applications with the Suppressed Electron Recombination”, Chem. Euro. J., 24 (68): 18032-18042 (2018)). Zhang et al. synthesized an acceptor-donor-acceptor H₂DPP using benzothiadiazole at the 2,5-aryl position to yield a chromophore exhibiting red emission with a solution fluorescence quantum yield (Φ) of 57% and a maximum external quantum efficiency (EQE) of 3.4% in an OLED device (Zhou, Y., et al., “Efficient Solution-Processed Red Organic Light-Emitting Diode Based on an Electron-Donating Building Block of Pyrrolo[3,2-b]Pyrrole”, Org. Electron., 65: 110-115 (2019)). Finally, H₂DPPs have been used in resistive memory devices and organic photodetectors (Canjeevaram, B., “Quadrupolar (A-π-D-π-A) Tetra-Aryl 1,4-Dihydropyrrolo[3,2-b]Pyrroles as Single Molecular Resistive Memory Devices: Substituent Triggered Amphoteric Redox Performance and Electrical Bistability”, J. Phys. Chem. C, 120 (21): 11313-11323 (2016). While these examples highlight the potential applicability of molecular H₂DPP materials, there is not an example of H₂DPPs being directly incorporated into a polymeric main chain, thus, fundamental structure-property relationships of polymer-containing H₂DPPs are unknown.

The present disclosure relates to uses of H₂DPP as a monomeric building block for synthesizing conjugated polymers. As previously discussed H₂DPP possesses easily tailored chromophores, combined with simple synthesis and purification techniques. The synthesis includes synthesizing a di-brominated H₂DPP. Di-brominating H₂DPPs enables the synthetically simple H₂DPP comonomer to further participate in Pd-catalyzed polymerizations. As non-limiting examples, synthetically simple monomers provide several advantages. At least, synthetically simple monomers reduce sime requirements and lower the environmental impact of synthesis. An additional, non-limiting advantage includes lowered production costs. The present disclosure relates to a novel incorporation of H₂DPPs into repeating units of the main chain of a polymer. Such polymer may be a copolymer. Additionally, the present disclosure relates to the use of monomers functionalized with side chains incorporated into the main chains of polymers.

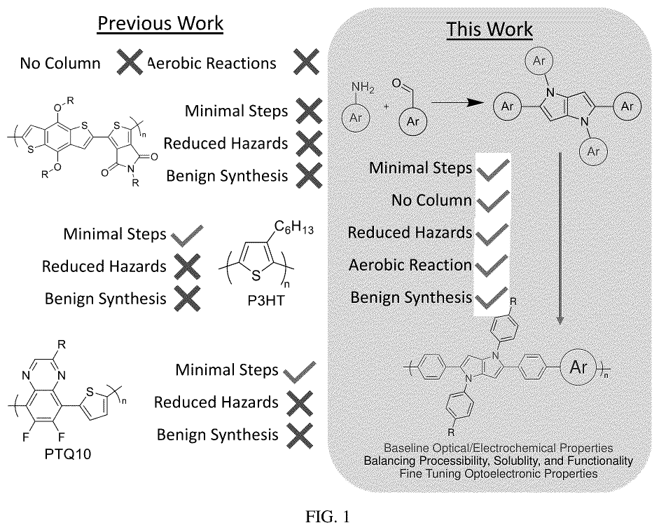

The present disclosure relates to the first use of an H₂DPP-containing copolymer synthesized via direct heteroarylation polymerization (DHAP) with 3,4-propylenedioxythiophene (ProDOT) as a co-monomer. The use of DHAP presents several advantages over the art. As non-limiting examples, DHAP at least reduces monomer preparation steps. This saves time and money by reducing the potential use of extra reagents and the potential generation of extra waste that would be need to be disposed. The present disclosure further relates to studies of structure-property relationships of this novel material by understanding how monomer structures influence synthetic accessibility, optical, thermal, and electrochemical properties. Additionally, the synthetic complexity that reveals the first H₂DPP copolymer to be amongst the simplest conjugated polymers to be synthesized is quantified. As shown in FIG. 1, the incorporation of H₂DPP into polymeric materials offers a readily accessible and tunable scaffold for conjugated polymers without compromising properties commonly associated with solid-state or electrochemical applications. The present disclosure relates to the ability to incorporate a synthetically simple monomer into the main chain of a polymer repeat unit while simultaneously providing a novel building block that may find utility in next-generation organic materials.

The present disclosure additionally relates to uses of an H₂DPP-containing copolymer synthesized via direct heteroarylation polymerization (DHAP) with other co-monomers. In such example, an H₂DPP-containing copolymer can be synthesized where an electron-deficient monomer serves as a co-monomer. Examples of electron-defficient co-monomers include but are not limited to diketopyrrolopyrrole (ketoDPP) and thienylpyrrolodione (TPD), as shown in FIG. 2.

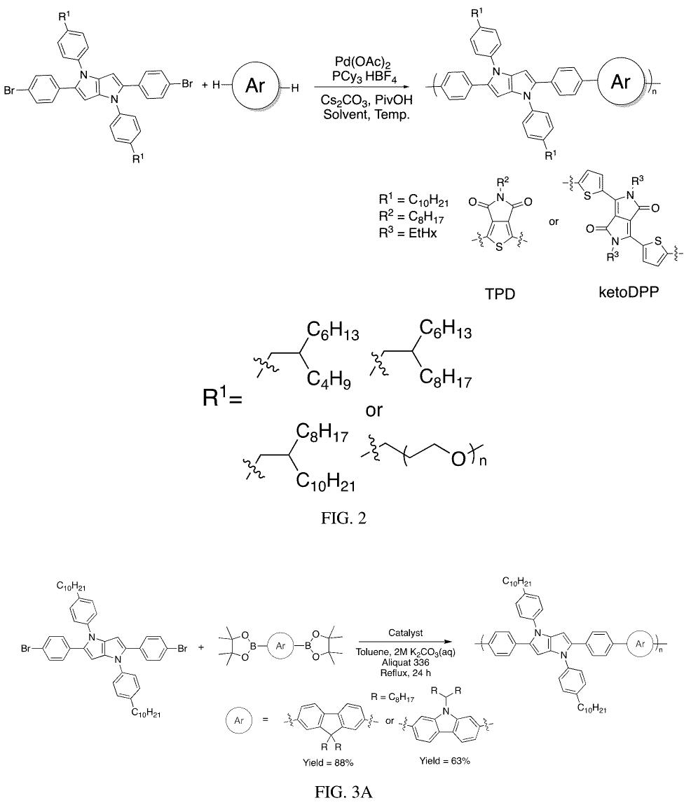

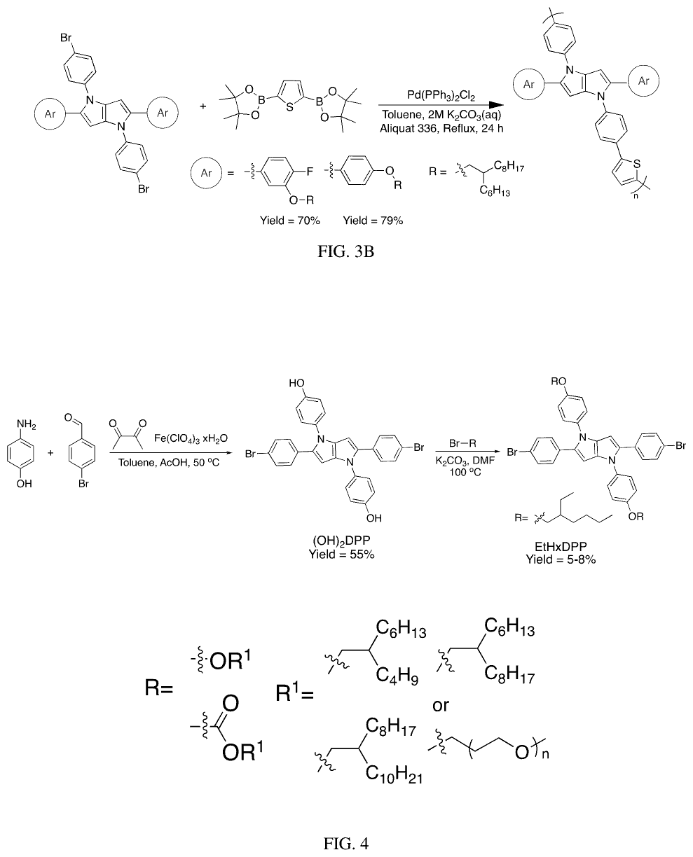

The present disclosure additionally relates to synthesis of H₂DPP copolymers via Suzuki poly-condensation reactions, as shown in FIGS. 3A-3B.

### III. Methods of Polymer Synthesis

The present disclosure relates to simplified methods of polymer synthesis. Polymers include high-performance conjugated polymers. Simplified methods of polymer synthesis address solubility concerns relating to conjugated polymers. In doing so, the simplified methods synthesize H₂DPP monomers that are capable of being used in soluble copolymers. Solubility concerns may arise where a conjugated polymer is not soluble. This may make synthesizing said conjugated polymer difficult where it is not amenable to high-throughput solution processing.

The present disclosure relates to approaches that eliminate or reduce process steps requiring air-free reactions, producing toxic byproducts, requiring chromatography for purification, and using high temperature/pressure or cryogenic reaction conditions. By eliminating such factors, synthesis is simplified. As a non-limiting example, reactions described herein may be conducted at temperatures ranging from about 20° C. up to about 200° C. In a particular non-limiting example, reactions described herein may be conducted at temperatures ranging from about 50° C. up to about 140° C.

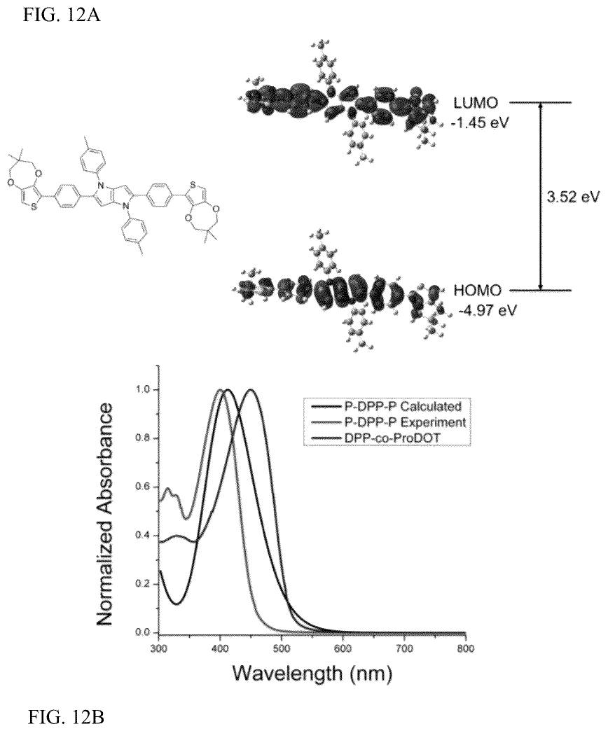

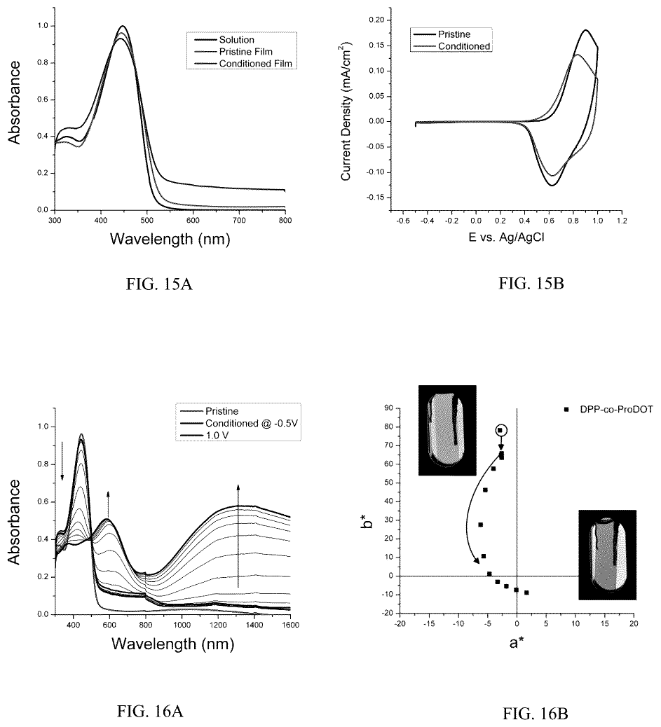

The present disclosure relates to simplified monomer and polymer synthetic steps. As a non-limiting example, the present disclosure relates to synthesis of a dibrominated dihydropyrrolopyrrole in air. In such an example, a single synthetic step is used that does not require column chromatography for purification. This shows the first example of a pyrrolopyrrole monomer being polymerized. Polymerization occurs via direct arylation polymerization using a dioxythiophene comonomer to access an alternating copolymer in a total of three synthetic steps. The resulting copolymer exhibits absorbance in the high-energy portion of the visible spectrum in solution and the solid state (FIG. 12B), a relatively low onset of oxidation (about 0.6 V vs Ag/AgCl) (FIG. 15B), and yellow-to-black electrochromism (FIG. 16B). The present disclosure relates to methods of measuring synthetic complexity. The synthetic complexity of pyrrolopyrrole-co-dioxythiophene polymer, as described herein, is less synthetically complex when compared to many conjugated polymers that find applicability in organic photovoltaics and electrochromism. These findings demonstrate the viability of incorporating dihydropyrrolopyrroles into the repeat unit structure of conjugated polymers via metal-catalyzed cross-coupling reactions. The present disclosure relates to the use of dihydropyrrolopyrroles as a synthetically simple alternative to current state-of-the-art conjugated polymers.

Conjugated polymers have traditionally been synthesized via Pd-catalyzed cross-coupling reactions, but these synthetic strategies suffer restrictive synthetic barriers in their respective procedures, such as monomer stability and potentially costly/toxic functionalization steps (Sakamoto, J., et al., “Polycondensation: Polyarylenes à La Carte”, Macromol. Rapid Commun. 30: 653-687 (2009); Carsten, B., et al., “Stille Polycondensation for Synthesis of Functional Materials”, Chem. Rev. 111 (3): 1493-1528 (2011)). The present disclosure improves upon these syntheses by minimizing the use/generation of toxic reagents. In doing so, direct heteroarylation polymerization (DHAP) is used to access synthetically simple conjugated polymers. DHAP is a green alternative polymerization strategy because it minimizes monomer preparation steps while simultaneously minimizing/eliminating toxic reagents by forgoing the need for organometallic reagents that participate in the transmetallation process of Pd-catalyzed cross-couplings (Mainville, M., et al., “Direct (Hetero)Arylation: A Tool for Low-Cost and Eco-Friendly Organic Photovoltaics”, ACS Appl. Polym. Mater, 3 (1): 2-13 (2021); Blaskovits, J. T. et al., “C—H Activation as a Shortcut to Conjugated Polymer Synthesis”, Macromol. Rapid Commun., 40 (1): 1800512 (2019); Pouliot, J. R., et al., “Direct (Hetero)Arylation Polymerization: Simplicity for Conjugated Polymer Synthesis”, Chem. Rev., 116 (22): 14225-14274 (2016); Pankow, R. M., et al., “The Development of Conjugated Polymers as the Cornerstone of Organic Electronics”, Polymer (Guildf), 207: 122874 (2020)). Additionally, DHAP is a robust polymerization strategy that enables accessing low-defect conjugated polymers without sacrificing device performance metrics (Ponder Jr, J. F., et al., “Low-Defect, High Molecular Weight Indacenodithiophene (IDT) Polymers Via a C—H Activation: Evaluation of a Simpler and Greener Approach to Organic Electronic Materials”, ACS Mater. Lett., 3 (10): 1503-1512 (2021)).

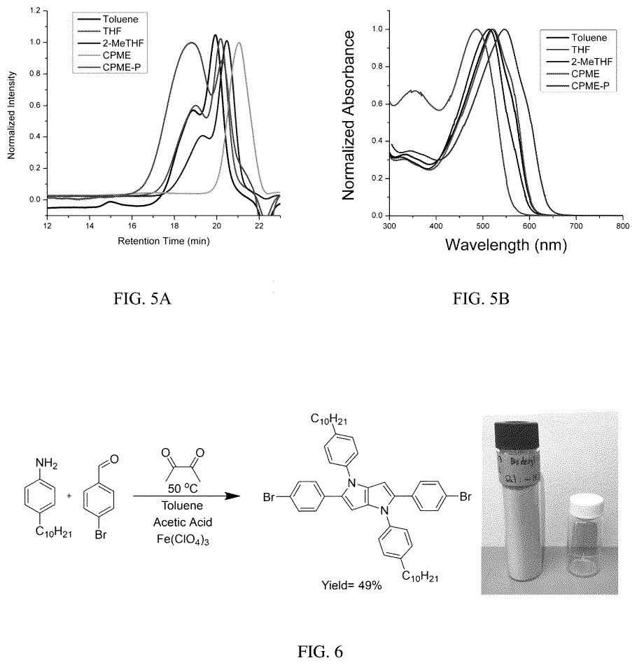

Initial polymerization attempts relating to the present disclosure to incorporate H2DPP as a comonomer into conjugated polymers were attempted with thienopyrroledione (TPD). TPD is a previously studied comonomer and shows a propensity to participate in DHAP (Pron, A., et al., “Thieno[3,4-c]Pyrrole-4,6-Dione-Based Polymers for Optoelectronic Applications”, Macromol. Chem. Phys., 214: 7-16 (2013)). Initial attempts were unsuccessful at obtaining copolymers with suitable solubility in organic solvents, evidenced by excessive material remaining in the Soxhlet thimble following extraction protocols in addition to bimodal molecular weight distributions measured via size-exclusion chromatography (SEC) (as shown in FIG. 5A). This ultimately prevented thorough and accurate characterization of polymeric properties and understanding of structure-property relationships, as shown in FIG. 5B. These limitations motivated pursuing alternative comonomers to overcome solubility barriers encountered in these initial studies.

The present disclosure relates to the use of 3,4-propylenedioxythiophene (ProDOT) as a comonomer for direct arylation polymerizations with H₂DPP. ProDOT offers facile tunability and synthetically simple monomers. This stems from its one-step synthesis and ease of functionalization for reactivity with numerous polymerization methods such as Grignard metathesis, oxidative polymerization, and Pd-catalyzed cross-couplings (Collier, G. S., et al., “Exploring the Utility of Buchwald Ligands for C—H Oxidative Direct Arylation Polymerizations”, ACS Macro Lett., 8 (8): 931-936 (2019)). Furthermore, various solubilizing motifs can be installed onto ProDOT in one or two steps via transetherification reactions that utilize commercially available starting materials, such as 2,2-di-n-octyl-1,3-propanediol (Reeves, B. D., et al., “Spray Coatable Electrochromic Dioxythiophene Polymers with High Coloration Efficiencies”, Macromolecules, 37 (20): 7559-7569 (2004)). Due to this tailoribility, ProDOT polymers have been used as electroactive material in various organic electronics such as electrochromics and OPVs (Dey, T., et al., “Poly(3,4-Propylenedioxythiophene)s as a Single Platform for Full Color Realization”, Macromolecules, 44 (8): 2415-2417 (2011); Mazaheripour, A., et al., “Nonaggregating Doped Polymers Based on Poly(3,4-Propylenedioxythiophene)”, Macromolecules, 52 (5): 2203-2213 (2019); Thompson, B. C., et al., “Soluble Narrow Band Gap and Blue Propylenedioxythiophene-Cyanovinylene Polymers as Multifunctional Materials for Photovoltaic and Electrochromic Applications”, J. Am. Chem. Soc., 128 (39): 12714-12725 (2006)). The use of ProDOT further provides the ability to understand structure-property relationships of H₂DPP-based copolymers.

The present disclosure relates to new monomers that efficiently participate in polymerization protocols while simultaneously lowering synthetic complexity and reducing toxicities associated with various reagents, pathways, and the like. Methods for minimizing synthetic steps while also removing toxic and air/moisture sensitive reagents during synthetic procedures are provided herein. The present disclosure displays the first example of an H₂DPP comonomer being directly incorporated into the main chain of a polymer repeat unit and providing foundational structure-property relationships of a novel class of polymeric material. As a non-limiting example, H₂DPP is used to create a H₂DPP-co-ProDOT copolymer.

As a non-limiting example of a synthesis, dibrominated H₂DPP comonomers are synthesized in one aerobic synthesis, purified via vacuum filtration, and are amendable to scalable preparations without sacrificing the purity required for efficient polymerizations. H₂DPP monomers are successfully incorporated into an electroactive conjugated polymer via direct arylation polymerization with a ProDOT comonomer, which enables studying monomer influence on properties such as degradation temperature, absorbance and fluorescence, and oxidation potential. Time-dependent density functional theory (TD-DFT) aids in explaining the wide bandgap absorbance features as well as the amorphous nature of the polymer in bulk and thin film samples by understanding the influence of dihedral angles on these properties. Electrochemical studies reveal the quasi-reversible redox nature of H₂DPP-co-ProDOT as a thin film that displays yellow-to-black electrochromism with a relatively low oxidation potential (about 0.6 V vs. Ag/AgCl).

H₂DPP successfully demonstrates the feasibility of generating electroactive materials while reducing the number of synthetic steps with relatively benign reagents. The present disclosure provides a new approach for simplifying the synthetic complexity commonly associated with generating conjugated polymers while also expanding the synthetic toolbox for the field of conjugated polymers.

### Aspects

The present disclosure is related to the following aspects.

A first aspect including a composition comprising a copolymer comprising a monomer derived from a pyrrole.

A second aspect including a composition of the first aspect, wherein the monomer is derived from a pyrrolopyrrole.

A third aspect including a composition of the second aspect, wherein the pyrrolopyrrole is pyrrolo[3,2-b]pyrrole (H₂DPP).

A fourth aspect including a composition of any of the above aspects further comprising a monomer derived from a dioxythiophene.

A fifth aspect including a composition of the fourth aspect, wherein the dioxythiophene is 3,4-propylenedioxythiophene (ProDOT).

A sixth aspect including a composition of any of the above aspects further comprising a monomer of thienopyrroledione (TPD) or diketopyrrolopyrrole (ketoDPP).

A seventh aspect including a compound comprising a copolymer comprising a repeating unit, wherein the repeating unit is of the following formula

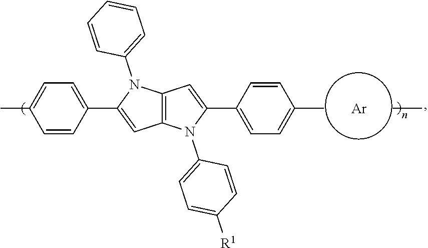

wherein R¹ comprises a functional group selected from the following formulas or C₁₀H₂₁

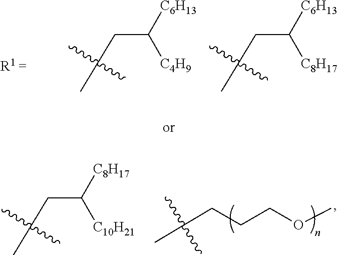

wherein Ar is an aromatic compound selected from the following formulas

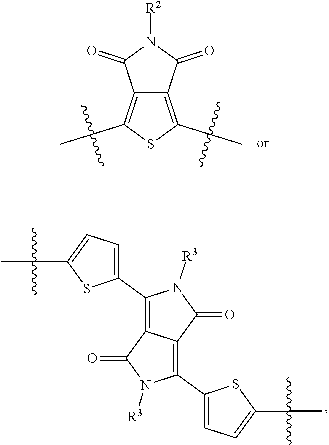

and wherein R² is C₈H₁₇ and R³ is EtHx.

An eighth aspect including a method of synthesizing a copolymer repeat unit derived from a pyrrole and a monomer derived from a dioxythiophene, the method comprising:
A. synthesizing a dihalogenated, pyrrole-derived monomeric unit;
B. synthesizing a dioxythiophene-derived monomeric unit;
C. synthesizing the copolymer through direct heteroarylation polymerization (DHAP) of the dibrominated, pyrrole-derived monomeric unit and the dioxythiophene-derived monomeric unit.

A ninth aspect including a method of the eighth aspect, wherein the pyrrole-derived monomeric unit is dibrominated.

A tenth aspect of incorporating at least one H₂DPP molecule into a repeat unit of a copolymer, the method comprising a reaction comprising:
A. providing a dibrominated, pyrrole-derived monomeric unit;
B. providing an aromatic compound; and
C. contacting the dibrominated, pyrrole-derived monomeric unit with the aromatic compound in a solution.

An eleventh aspect including a method of the tenth aspect, wherein the dibrominated, pyrrole-derived monomeric unit is of the following formula

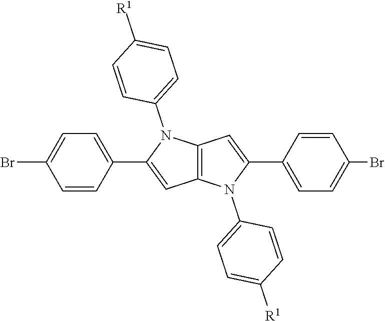

and wherein R¹ comprises a functional group selected from the following formulas or C₁₀H₂₁

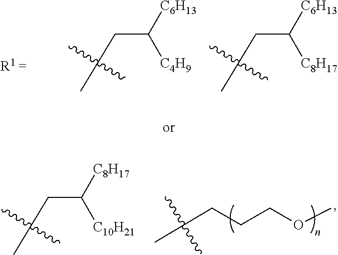

A twelfth aspect including a method of the tenth or eleventh aspect, wherein the aromatic compound is selected from the following formulas

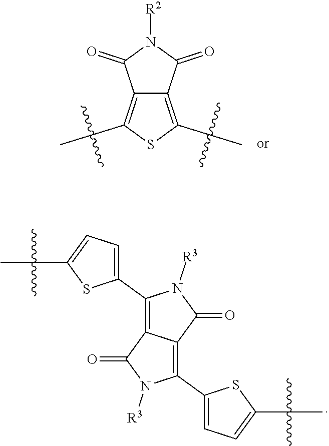

and wherein R² is C₈H₁₇ and R³ is EtHx.

A thirteenth aspect including a method of any of the tenth, eleventh, or twelfth aspect, wherein the solution comprises Pd(OAc)₂ and PivOH.

A fourteenth aspect including a method of the thirteenth aspect, wherein the solution further comprises PCy₃, HBF₄, and Cs₂CO₃.

A fifteenth aspect including a method of the thirteenth or fourteenth aspect, wherein the solution further comprises DMAc.

A sixteenth aspect including a method of any of aspects ten through fifteen, wherein the reaction is carried out at a temperature from about 50° C. up to about 140° C.

A seventeenth aspect including a method of any of aspects ten through sixteen, wherein the reaction is carried out at a temperature of about 140° C.

### EXAMPLES

### Example 1

### Methods

Copolymers were synthesized using Suzuki reactions, as shown in FIG. 3A. Br₂DHPP (251.1 mg, 0.295 mmol), the corresponding aryl-boronic ester (0.295 mmol), and 2 mol % of a palladium source (0.0589 mmol) (non-limiting examples of Pd sources provided in Table 1 below) were added to a 10 mL, one-neck round bottom flask along with a Teflon stir bar. One drop of Aliquat 336 was subsequently added to the flask. A condenser was connected to the round bottom flask and sealed with a rubber septum. The flask was rendered inert via vacuum/refill cycles with Ar three times before adding 2M K₂CO₃ (aq) and toluene via cannula (ratios found in Table 1 below). The reaction mixture was placed in an oil bath set to 110° C. and stirred overnight. The next day, the reaction mixture was cooled to room temperature before precipitation into about 200 mL of MeOH while vigorously stirring. The precipitate was collected in a Soxhlet thimble and then washed with MeOH and acetone to remove impurities and low molecular weight oligomers before extracting the desired polymer from the Soxhlet thimble with chloroform. The product was concentrated via rotary evaporation and precipitated into about 200 mL of MeOH while stirring. The precipitate was allowed to stir for 1 h before collecting the product via vacuum filtration. The polymer was placed into a vial and allowed to dry under vacuum overnight. Both polymers were confirmed to be the expected product after structural analysis.

TABLE 1
Data from efforts to optimize Suzuki polycondensation reaction conditions.

| | | Solvent/Base | | Yield | Mₙ | Dispersity |
|---|---|---|---|---|---|---|
| Entry | Comonomer | Ratio | Pd Source | (%) | (kg/mol) | (M_{w}/Mₙ) |
| 1 | FL | 1:1 | Pd(PPh₃)₄ | 81 | 9.17 | 1.6 |
| 2 | FL | 1:1 | Pd(PPh₃)₂Cl₂ | 53 | 13.0 | 6.6 |
| 3 | FL | 2:1 | Pd(PPh₃)₂Cl₂ | 59 | 7.10 | 8.2 |
| 4 | FL | 4:1 | Pd(PPh₃)₂Cl₂ | 33 | 14.0 | 3.7 |
| 5 | FL | 4:1 | ^{a}Pd(PPh₃)₄ | 88 | 10.6 | 4.8 |
| 6 | Car | 1:1 | Pd(PPh₃)₂Cl₂ | 50 | 7.85 | 1.8 |
| 7 | Car | 4:1 | ^{a}Pd(PPh₃)₄ | 63 | 7.88 | 1.8 |

^{a}Purified via vacuum filtration and washing with MeOH

### Results

Analytical results of the products obtained above are provided. 2,5-bis(phenyl)-1,4-bis(4-n-decylphenyl)-1,4-dihydropyrrolo[3,2-b]pyrrole-co-9,9-Dioctylfluorene (DHPP-co-FL): Brown Solid. Yield: 279 mg (88%) 1H NMR (400 MHz, CDCl3), δ: 0.70 (s, 3H), 0.79 (t, 9H), 0.88 (t, 9H), 1.05-1.19 (m, 28H), 1.28-1.39 (m, 41H), 1.63-1.70 (m, 6H), 2.02 (s, 3H), 2.66 (t, 5H), 6.48 (s, 2H), 6.48 (s, 2H), 7.19-7.35 (m, 18H), 7.55-7.62 (m, 8H), 7.72-7.77 (m, 2H). Anal. calc'd for C81H106N2: C 87.83; H 9.65; N 2.53 Found: C 87.59; H 9.38; N 2.62. Mₙ=10.6 kg/mol M_{w}=51.1 kg/mol M_{w}/Mₙ=4.8.

2,5-bis(phenyl)-1,4-bis(4-n-decylphenyl)-1,4-dihydropyrrolo[3,2-b]pyrrole-co-9-Heptadecan-9-ylcarbazole (DHPP-co-Car): Dark-Brown Solid. Yield: 213.5 mg (63%) 1H NMR (400 MHz, CDCl3), δ: 0.77-0.83 (m, 9H), 0.87 (t, 9H), 1.13-1.40 (m, 90H), 1.63-1.70 (m, 7H), 1.96 (s, 2H), 1.96 (s, 2H), 2.35 (2H), 2.65 (t, 5H), 4.64 (s, 2H), 6.50 (s, 2H), 7.19-7.25 (m, 8H), 7.33 (dd, J=23.4, 8.1 Hz, 11H), 7.46 (d, 4H), 7.58-7.61 (m, 6H), 7.70-7.75 (m, 2H). Anal. calc'd for C81H107N3: C 86.65; H 9.61; N 3.74 Found: C 85.17; H 9.12; N 3.79. Mₙ=7.9 kg/mol M_{w}=14.4 kg/mol M_{w}/Mₙ=1.8.

### Example 2

### Methods

Additional copolymers were synthesized using Suzuki reactions, as shown in FIG. 3B. Br₂-F,ORDHPP (200.4 mg, 0.184 mmol) or Br₂-4-ORDHPP (200.3 mg, 0.191 mmol), thiophene-2,5-diboronic acid bis(pinacol) ester (0.184 or 0.191 mmol), and 2 mol % of bis(triphenylphosphine)palladium(II)chloride (Pd(PPh3)2Cl2) (4.2 mg, 0.0589 mmol) were added to a 10 mL one-neck round bottom flask along with a Teflon stir bar. One drop of Aliquat 336 was subsequently added to the flask. The round bottom flask was fitted with a condenser and was sealed with a rubber septum. The flask was rendered inert via vacuum/refill cycles with Ar (3×) before adding 1 mL of 2M K₂CO₃ (aq) and 4 mL of toluene to the flask via cannula. The reaction mixture was placed in an oil bath thermostatted at 110° C. and allowed to stir overnight. The reaction mixture was cooled to room temperature before precipitation into about 200 mL of MeOH while vigorously stirring. The precipitate was collected in a cellulose Soxhlet thimble and then washed with methanol and acetone to remove impurities and low molecular weight oligomers before extraction of the desired polymer with chloroform. The product was concentrated via rotary evaporation and precipitated into about 200 mL of MeOH while stifling. The polymer/methanol solution was allowed to stir for 1 h before collecting the product via vacuum filtration. The polymer was placed into a vial and allowed to dry on the vacuum overnight. Both polymers were confirmed to be the expected product after structural analysis (vide infra).

### Results

Analytical results of the products obtained above are provided. 2,5-bis(4-fluoro-3-hexyldecyletherphenyl)-1,4-bis(phenyl)-1,4-dihydropyrrolo[3,2-b]pyrrole-co-thiophene (F,ORDPP-co-Th): Pale-yellow solid. Yield: 133 mg (70%). 1H NMR: (400 MHz, CDCl3), δ: 0.87-0.92 (m, 15H), 1.26-1.40 (m, 62H), 1.68-1.71 (m, 3H), 3.62-3.70 (m, 4H), 6.45-6.46 (m, 2H), 6.76-6.86 (m, 4H), 6.95-7.02 (m, 3H), 7.32-7.35 (m, 5H), 7.68 (d, J=8.3 Hz, 4H). Anal. calc'd for C66H84F2N2O2S Theory: C, 78.53; H, 8.59; N, 2.78; S, 3.18 Actual: C, 77.57; H, 8.36; N, 2.86; S, 2.87. Mₙ=16 kg/mol M_{w}=67 kg/mol M_{w}/Mₙ=4.2.

2,5-bis(4-hexyldecyletherphenyl)-1,4-bis(phenyl)-1,4-dihydropyrrolo[3,2-b]pyrrole-co-thiophene (4-ORDPP-co-Th): Dark-brown solid. Yield: 146 mg (79%). 1H NMR: (400 MHz, CDCl3), δ: 0.88-0.92 (m, 15H), 1.29-1.49 (m, 62H), 1.76-1.81 (m, 3H), 3.84 (d, 4H), 6.34-6.41 (m, 2H), 6.79-6.85 (m, 4H), 7.07-7.23 (m, 6H), 7.30-7.35 (m, 5H), 7.49 (d, J=8.5 Hz, 1H), 7.61-7.66 (m, 4H). Anal. calc'd for C66H84N202S Theory: C, 81.43; H, 9.11; N, 2.88; S, 3.29 Actual: C, 79.51; H, 8.67; N, 2.89; S, 2.83. Mₙ=7.1 kg/mol M_{w}=9.7 kg/mol M_{w}/Mₙ=1.4.

### Example 3

### Methods

Efforts to address solubility concerns relating to simplified synthesis of high-performance conjugated polymers began with attempting to synthesize H₂DPP using 4-aminophenol and 4-bromobenzaldehyde followed by alkylation protocol (Wang, J., et al., “Organic Dyes Based on Tetraaryl-1,4-Dihydropyrrolo[3,2-b]Pyrroles for Photovoltaic and Photocatalysis Applications with the Suppressed Electron Recombination”, Chem. Euro. J., 24 (68): 18032-18042 (2018)), as shown in FIG. 4.

### Results

(OH)2DPP was obtained with a favorable yield of 55%. Subsequent alkylation attempts via Williamson etherification resulted in yields between 5-8%.

### Example 4

### Methods

In an effort to improve yields of alkylation via Williamson etherification, and overall atom economy, 4-aminophenol was replaced with 4-n-decylaniline to access a monomer amendable to Pd-catalyzed polymerizations in one-step, in air, and requiring simple purification(s) (e.g. vaccum filtration). 4-Decylaniline was chosen as it was hypothesized to be a suitable solubilizing handle for the resulting polymers, thus enabling thorough characterization and eventually solution proces sibility.

### Results

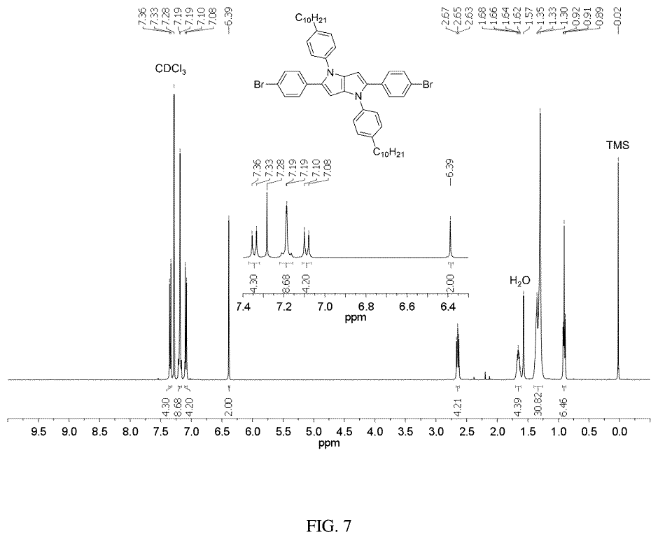

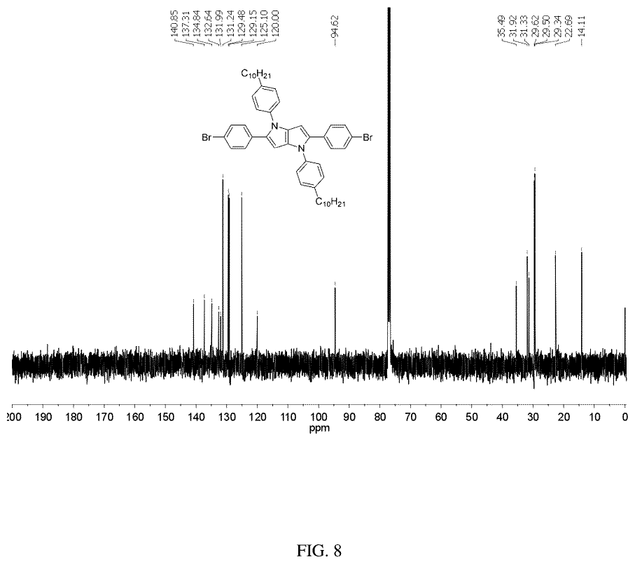

As shown in FIG. 6, this change improved the overall yield of the desired monomer from 8% to 49%. The structure and purity of the resulting monomer were verified via ¹H and ¹³C NMR (FIGS. 6-7) and elemental analysis, respectively. After a successful synthesis, the reaction was scaled to 80 mmol to save time on future synthetic efforts as well as to simulate potential industrial scalability. Notably, the product from the 80 mmol reaction does not sacrifice the yield nor purity of the resulting monomer compared to the same monomer isolated from smaller scale reactions and yielded about 15 grams in a single synthetic step.

### Example 5

### Methods

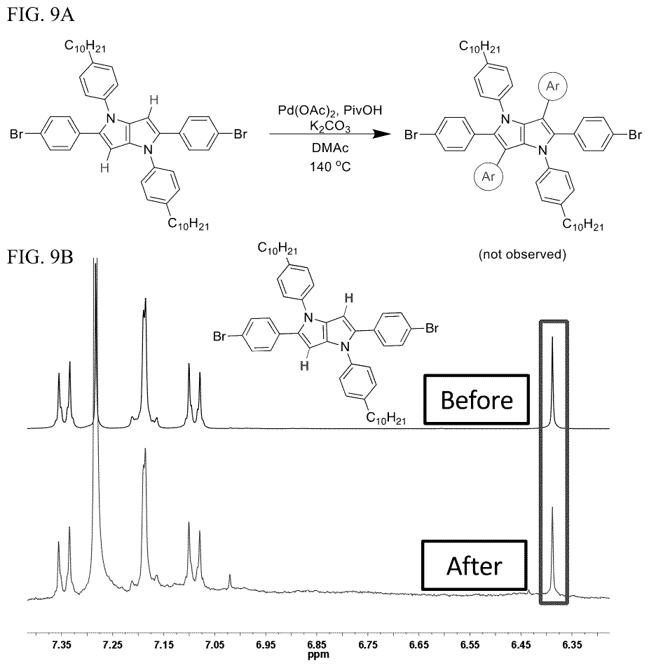

In order to polymerize H₂DPP with ProDOT, an established DHAP procedure for producing poly(ProDOT)s that uses the high dielectric solvent N,N-dimethylacetamide (DMAc), palladium(II) acetate (Pd(OAc)2) as the catalyst, potassium carbonate (K2CO3) as the base, and pivalic acid (PivOH) as the proton shuttle while heating the reaction mixture at 140° C. was used (Estrada, L. A., et al., “Direct (Hetero)Arylation Polymerization: An Effective Route to 3,4-Propylenedioxythiophene-Based Polymers with Low Residual Metal Content”, ACS Macro Lett., 2 (10): 869-873 (2013)). The 3,6-position hydrogens of H₂DPP, illustrated as the red protons in FIG. 9A, have been shown to participate in direct arylation reactions to access multi-aryl H₂DPP chromophores (Krzeszew ski, M., et al., “Tetraaryl-, Pentaaryl-, and Hexaaryl-1,4-Dihydropyrrolo[3,2-b]Pyrroles: Synthesis and Optical Properties”, J. Org. Chem., 79 (7): 3119-3128 (2014); Banasiewicz, M., et al., “Electronic Communication in Pyrrolo[3,2-b]Pyrroles Possessing Sterically Hindered Aromatic Substituents”, Eur. J. Org. Chem., 2019 (31-32): 5247-5253 (2019); Ryu, H. G., et al., “Bidirectional Solvatofluorochromism of a Pyrrolo[3,2-b]Pyrrole-Diketopyrrolopyrrole Hybrid”, J. Phys. Chem., 122 (25): 13424-13434 (2018)). This motivated screening the H₂DPP monomer in high dielectric DHAP conditions and monitoring for any observable coupling at the 3,6-positions of the pyrrolopyrrole scaffold. Such coupling at the 3,6-position would result in cross-linked polymer or polymers with an appreciable number of β-defects, which may produce insoluble materials or lead to diminished material properties (Sadowski, B., et al., “Tetraphenylethylenepyrrolo[3,2-b]Pyrrole Hybrids as Solid-State Emitters: The Role of Substitution Pattern”, Org. Lett., 20 (11): 3183-3186 (2018); Hatanaka, S., et al., “Tris(Pentafluorophenyl)Borane-Pyrrolo[3,2-b]Pyrrole Hybrids: Solid-State Structure and Crystallization-Induced Enhanced Emission”, ChemPhotoChem, 4 (2): 138-143 (2020). After subjecting the pyrrolopyrrole monomer to the reaction conditions mentioned above, the first indication of the absence of cross-coupling defects was from not observing any new spots on thin layer chromatography (TLC). These results were then confirmed with ¹H NMR (vide infra).

### Results

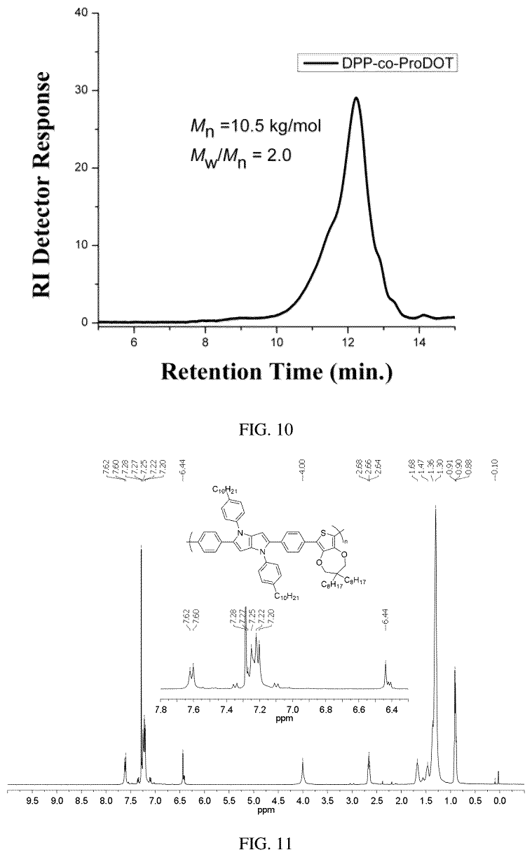

FIG. 9B shows overlaid ¹H NMR spectra of Br₂DPP before and after the model reaction. As shown in FIG. 9B, the lack of change for the singlet at about 6.37 ppm as well as the absence of new chemical shifts in the aromatic region confirms no 3,6-coupling was observed and encourages the pursuit of polymerizations using the high dielectric conditions. The established DHAP procedure yielded a yellow polymer with a number-average molecular weight (Mₙ) of 10.5 kg/mol and dispersity (M_{w}/Mₙ) of 2.0, as determined by SEC versus polystyrene standards using CHCl₃ as the eluent (FIG. 10). Polymer solutions were difficult to filter through a 0.45 μm SEC prefilter, which may have inhibited the accurate estimation of the polymer molecular weight. Nonetheless, polymers with a Mₙ>10 kg/mol are typically considered of sufficient molecular weight such that electronic properties are saturated and result in polymers with appropriate viscosities needed for solution processing of thin films (Thompson, B. C., et al., “Soluble Narrow Band Gap and Blue Propylenedioxythiophene-Cyanovinylene Polymers as Multifunctional Materials for Photovoltaic and Electrochromic Applications”, J. Am. Chem. Soc., 128 (39): 12714-12725 (2006)). The resulting polymer retained the pyrrolopyrrole singlet at 6.38 ppm and the singlet at 4.10 ppm, attributed to protons on the propylene bridge of ProDOT, thus confirming successful incorporation of ProDOT into the polymer in an alternating manner (see FIG. 11). Purity and composition were further verified with elemental analysis that showed similar values for expected and determined atomic compositions. Polymers with high compositional purity are required for accurately determining structure-property relationships as residual impurities are known to be detrimental to the performance of organic materials (Usluer, Ö., et al., “Metal Residues in Semiconducting Polymers: Impact on the Performance of Organic Electronic Devices”, ACS Macro Lett., 3(11): 1134-1138 (2014); Curtin, I. J., et al., “Role of Impurities in Determining the Exciton Diffusion Length in Organic Semiconductors”, Appl. Phys. Lett., 108 (16): 163301 (2016); Bracher, C., et al., “The Effect of Residual Palladium Catalyst on the Performance and Stability of PCDTBT:PC70BM Organic Solar Cells”, Org. Electron., 27: 266-273 (2015)).

### Example 6

### Methods

Optical properties of H₂DPP-co-ProDOT described herein were quantified using UV-vis and fluorescence spectroscopies. After successful synthesis, calculations to probe fundamental structural and optical properties were made. Excited state transitions of a (ProDOT)₂DPP oligomer were calculated via time-dependent density functional theory (TD-DFT) using the mPW1PBE functional paired with the cc-PVDZ basis set. These parameters were chosen because they have been shown to accurately correlate calculated and experimental absorbance spectra of ProDOT-containing oligomers (Wheeler, D. L., et al., “Modeling Electrochromic Poly-Dioxythiophene-Containing Materials Through TDDFT”, Phys. Chem. Chem. Phys., 19 (30): 20251-20258 (2017); Collier, G. S., et al., “Exploring Isomeric Effects on Optical and Electrochemical Properties of Red/Orange Electrochromic Polymers”, Macromolecules, 54 (4): 1677-1692 (2021)).

### Results

FIG. 12A and Table 1 below summarize the results showing that model oligomers possess wide band gaps (about 3.5 eV) likely due to the large dihedral angles between the H₂DPP-Ph units (about 35°).

TABLE 2

| HOMO, 212 (eV) | LUMO, 213 (eV) |
|---|---|
| −4.97 | −1.45 |

BAND GAP (eV)
3.00

| | | | Dihedral | Dihedral |
|---|---|---|---|---|
| | | | (DPP-Phenylene) | (Phenylene-Ar) |
| MPW1PBE/SV | | | 35.32 | 19.30 |
| | | eV | λ (nm) | f |
| | ES1 | 3.00 | 413 | 1.8623 |
| | H→L | 0.70 | | |
| | ES12 | 4.39 | 283 | 0.5246 |
| | H-3→L | 0.50 | | |
| | ES14 | 4.64 | 267 | 0.2658 |
| | H→L + 2 | 0.62 | | |
| | H→L + 4 | 0.26 | | |

| MO | | | Eigenvalues (eV) |
|---|---|---|---|
| | Occ: H-4 | 208 | −6.69526655 |
| | Occ: H-3 | 209 | −6.37063932 |
| | Occ: H-2 | 210 | −5.96383487 |
| | Occ: H-1 | 211 | −5.75757549 |
| | Occ: H | 212 | −4.96519117 |
| | Unocc: L | 213 | −1.45415584 |
| | Unocc: L + 1 | 214 | −1.07619505 |
| | Unocc: L + 2 | 215 | −0.36870905 |
| | Unocc: L + 3 | 216 | −0.27374266 |
| | Unocc: L + 4 | 217 | −0.26476303 |

Calculated dihedral angles were consistent with previous electronic structure calculations as well as dihedral angles found for published H₂DPP crystal structures, thus supporting the level of theory (Banasiewicz, M., et al., “Electronic Communication in Pyrrolo[3,2-b]Pyrroles Possessing Sterically Hindered Aromatic Substituents”, Eur. J. Org. Chem., 2019 (31-32): 5247-5253 (2019); Sadowski, B., et al., “Tetraphenylethylenepyrrolo[3,2-b]Pyrrole Hybrids as Solid-State Emitters: The Role of Substitution Pattern”, Org. Lett., 20 (11): 3183-3186 (2018)). The frontier molecular orbital maps (FIG. 12A) displayed the electron density of the HOMO resides mostly on the H₂DPP unit with modest redistribution to ProDOT in the LUMO. This result supports the notion of H₂DPP being a highly electron-rich building block (Tanaka, S., et al., “1,4-Dihydropyrrolo[3,2-b]Pyrrole: The Electronic Structure Elucidated by Photoelectron Spectroscopy”, Bull. Chem. Soc. Jpn., 60 (6): 1981-1983 (1987)). Calculated UV-vis absorbance spectra were blue-shifted compared to H₂DPP-co-ProDOT, as shown in FIG. 12B. A (ProDOT)₂DPP oligomer was synthesized to compare theory and experimental results for this H₂DPP system. FIG. 12B shows the overlaid UV-vis spectra for calculated, experimental, and H₂DPP-co-ProDOT, where calculated and experimental oligomer UV-vis data were in close agreement (about 2% λₘₐₓ). These results further support the level of theory and indicated the effective conjugation length of H₂DPP-co-ProDOT is greater than 5 rings even with a large amount of torsional strain distributed through the polymer backbone.

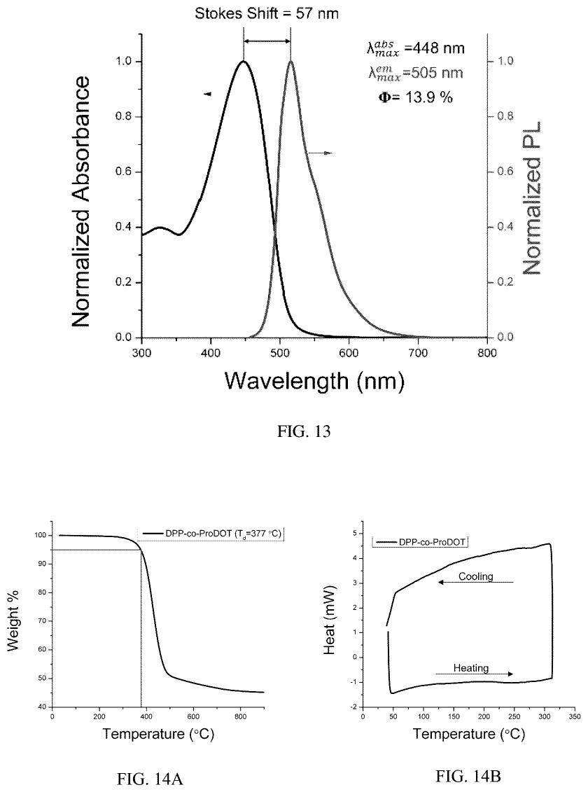

As shown in FIG. 13, H₂DPP-co-ProDOT absorbed with a λₘₐₓ^{abs} of 448 nm, which is attributed to the π-π* transition. The featureless absorbance indicated a lack of aggregates present in solution, suggesting the polymer is well solvated in toluene at this concentration. Upon photoexcitation, H₂DPP-co-ProDOT emitted green light with a λₘₐₓ^{PL} of 505 nm. A Stokes shift of 57 nm revealed a modest degree of structural rearrangement upon photoexcitation, as shown in FIG. 12. Since H₂DPP chromophores have been shown to possess high fluorescence quantum yields, the solution quantum yield (Φ) of H₂DPP-co-ProDOT was measured to be 13.9% in toluene, indicating a modest fluorescence quantum yield. Quantum yields of Φ≥10% are typically viewed as sufficient for use in organic light-emitting diodes (Ravindran, E., et al., “Efficient White-Light Emission from a Single Polymer System with “Spring-like” Self-Assemblies Induced Emission Enhancement and Intramolecular Charge Transfer Characteristics”, J. Mater. Chem. C, 5 (19): 4763-4774 (2017)). H₂DPP-co-ProDOT exhibited signs of photo-oxidation in solution under ambient conditions after prolonged exposure to sunlight (e.g. two or more weeks). This phenomenon is accelerated for polymers dissolved in chlorinated solvents when irradiated with UV light. Additionally, this photo-oxidation was attributed to the electron-rich properties of the H₂DPP core in the polymer facilitating a photoinduced electron transfer (PET) process that has been reported for H₂DPP chromophores used for detecting halocarbons (Wu, J.Y., et al., “Pyrrolo-[3,2-b]Pyrroles for Photochromic Analysis of Halocarbons”, Anal. Chem., 88 (2): 1195-1201 (2016)).

### Example 7

### Methods

Initial understanding of H₂DPP-co-ProDOT thermal properties, such as degradation temperature (T_{d}), glass transition (T_{g}), and crystallization temperature (Tₐ), were studied using thermal gravimetric analysis (TGA) and differential scanning calorimetry (DSC), respectively. TGA was used to determine the degradation temperature of H₂DPP-co-ProDOT by measuring the mass loss as a function of temperature.

### Results

FIG. 14A shows thermal gravimetric analysis (TGA) of H₂DPP-co-ProDOT ramping from 30-900° C. at a rate of 10° C./min. The TGA trace that indicates H₂DPP-co-ProDOT had a T_{d} of 377° C. while also providing insight into the purity of the resulting polymer. The mass loss at T_{d} is consistent with the loss of solubilizing side chains from H₂DPP and ProDOT. The absence of degradation and the level trace from r.t. to about 300° C. indicated there were no residual solvents or salts in the polymer and supported NMR and elemental analysis results (Lo, C. K., et al., “From Monomer to Conjugated Polymer: A Perspective on Best Practices for Synthesis”, Chem. Mater., 33 (13): 4842-4852 (2021)). FIG. 14B shows differential scanning calorimetry (DSC) trace of H₂DPP-co-ProDOT (second heating cycle) cycling between 30-310° C. with a heating rate of 10° C./min. DSC measurements showed no thermal transitions within the experimental range, revealing that the polymer was amorphous in bulk samples, as shown in FIG. 14B. This was attributed to the large dihedral angles between the pyrrolopyrrole core and phenylene rings that prevented strong interactions between polymer chains that encourage a higher degree of order and crystallinity. Because H₂DPP-co-ProDOT possessed a high T_{d}, it is reasonable to surmise that this polymer can be solution processed and subjected to post-processing annealing procedures, if needed.

### Example 8

### Methods

Due to the electron-rich nature of H₂DPP and ProDOT, redox behavior of H₂DPP-co-ProDOT as thin films was studied. First, films were spray-cast from 2 mg/mL toluene on to ITO electrodes.

### Results

When the UV-vis absorbance spectrum of the resulting film was measured, there was minimal change in the absorbance profile from solution to a pristine film. Typically, conjugated polymers exhibit a distinct red-shift in the absorbance spectrum in the solid state when compared to solution due to an increase in π-πinteractions that facilitate ordering. The lack of change in the absorbance spectrum indicated minimal interchain π-πorbital overlap in the solid state. This was attributed to the large dihedral angles through the polymer backbone that prevented efficient interchain polymer interactions.

### Example 9

### Methods

After comparing the optical properties of the H₂DPP-co-ProDOT copolymer in solution and as a pristine film, films were electrochemically conditioned to study the optical properties after subjecting the films to repeated redox reactions. Electrochemical conditioning was necessary because redox-active polymers often display distinct changes in their redox response and optical properties with repeated exposure to redox reactions (Heinze, J., et al., “Electrochemistry of Conducting Polymers—Persistent Models and New Concepts”, Chem. Rev., 110 (8): 4724-4771 (2010)). Electrochemical conditioning protocols consisted of performing 10 cyclic voltammetry (CV) cycles across a voltage window of −0.5 V to 1.0 V (vs. Ag/AgCl reference electrode) in a 0.5 M TBAPF₆/PC electrolyte solution using a scan rate of 100 mV/s.

### Results

Upon electrochemical conditioning, there was only a slight broadening of the absorbance profile and the λₘₐₓ was unchanged, as shown in FIGS. 14A-14B. This was a rare observation since conjugated polymers typically exhibit distinct changes in the absorbance spectrum after being subjected to a flux of solvent and ions through the film. This supported the notion that the polymer was in a disordered state and electrochemical conditioning did not lead to a change in the effective conjugation length, which was attributed to the observed changes in the absorbance spectrum of conjugated polymers after repeated redox cycling (Collier, G. S., et al., “Exploring Isomeric Effects on Optical and Electrochemical Properties of Red/Orange Electrochromic Polymers”, Macromolecules, 54 (4): 1677-1692 (2021)).

### Example 10

### Methods

Next, the electrochemical properties were studied using cyclic voltammetry (CV).

### Results

As shown in FIG. 15B, despite H₂DPP-co-ProDOT possessing large dihedral angles distributed through the backbone, the polymer displayed a relatively low onset of oxidation (about 0.6 V vs Ag/AgCl). This redox response was analogous to arylene-based copolymers used to access high-gap electrochromic polymers (Kerszulis, J. A., et al., “Follow the Yellow Brick Road: Structural Optimization of Vibrant Yellow-to-Transmissive Electrochromic Conjugated Polymers”, Macromolecules, 47 (16): 5462-5469 (2014); Amb, C. M., et al., “Propylenedioxythiophene (ProDOT)-Phenylene Copolymers Allow a Yellow-to-Transmissive Electrochrome”, Polym. Chem., 2 (4): 812-814 (2011)). but lower than triphenylamine-based copolymers (Yen, H. J., et al., “ Solution-Processable Triarylamine-Based Electroactive High Performance Polymers for Anodically Electrochromic Applications”, Polym. Chem., 3 (2): 255-264 (2012); Beaupré, S., et al., “Toward the Development of New Textile/Plastic Electrochromic Cells Using Triphenylamine-Based Copolymers”, Chem. Mater., 18 (17): 4011-4018 (2006); Jeong, J., et al., “Synthesis and Characterization of Triphenylamine-Based Polymers and Their Application Towards Solid-State Electrochromic Cells”, RSC Adv., 6 (82): 78984-78993 (2016)). Additionally, the minimal change in absorbance from pristine to conditioned film was confirmed with CV. As seen in FIG. 15B, H₂DPP-co-ProDOT displayed a quasi-reversible oxidation and reduction both as pristine and conditioned film, where the onset of oxidation was slightly lower in the conditioned film (about 0.6 V vs Ag/AgCl). In contrast, the reduction peak was unchanged after electrochemical conditioning. Another observation was the increased slope of the CV trace between pristine and conditioned films when reducing the film from 1.0 to 0.8 V. This increase was indicative of solvent and ions moving through the film more rapidly due to more accessible redox sites being available.

Absorbance as a function of electrochemical potential was studied, and the results are plotted in FIG. 16A. As shown, with increasing electrochemical potential, the π-π* transition about 448 nm decreased and two new transitions evolved. According to simulation results, both were due to the formation of a radical cation (Heimel, G., “The Optical Signature of Charges in Conjugated Polymers”, ACS Cent. Sci., 2 (5): 309-315 (2016)), with one species broadly absorbing across the visible spectrum and the second in the infrared (IR) portion of the electromagnetic spectrum with a maximum absorbance about 1300 nm. Both grew continuously with increasing electrochemical potential.

### Example 11

### Methods

Colorimetric analysis of polymer films was subsequently performed based on the “Commision Internationale de l'Eclairage” 1976 L*a*b* color standards.

### Results

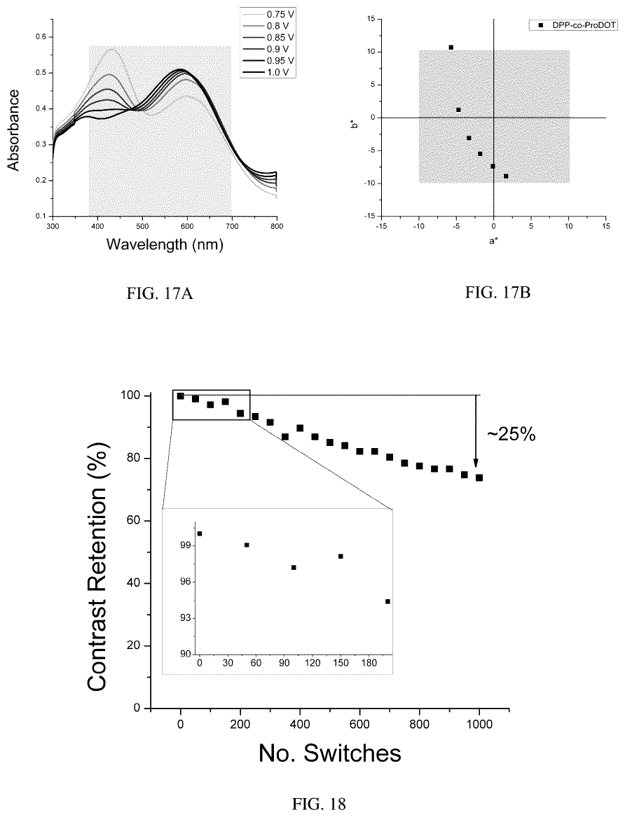

Colorimetry data shown in FIG. 16B indicated H₂DPP-co-ProDOT as a vibrant yellow color as a neutral film, with a large b* (about 68) and small a* (about −3), which agreed with the absorbance in the high energy portion of the visible spectrum (400-500 nm). Additionally, colorimetry confirmed the minimal changes between pristine (circled) and electrochemically conditioned films observed in the UV-vis absorbance spectrum as evidenced by only a small decrease in the b* value while the a* value remained constant. As the oxidation potential increased, the b* values began to decrease, which corresponded to the evolution of the broadly absorbing oxidized species. While b* values decreased, a* values tracked slightly more negative before returning towards the graph's origin. This color track towards the green portion of the color space was due to the pseudo dual-band absorbance character between the diminishing π-π* transition and evolving absorbance about 600 nm. As the broad absorbance features continued to evolve (FIG. 17A), a* and b* coordinates tracked to the color-neutral portion of the a*b* plot (FIG. 17B).⁶⁶ The color neutrality manifested itself as a black film in the oxidized state, as shown in the photograph presented in FIG. 16B.

### Example 12

### Methods

The long-term electrochemical stability was measured by applying square-wave potential steps from −0.5 V to 1.0 V vs Ag/AgCl to switch films under an argon atmosphere.

### Results

As shown in FIG. 18, H₂DPP-co-ProDOT films maintained about 95% of contrast after 200 switching cycles but lost 25% of contrast retention after 1000 cycles. This was likely caused by open sites on the phenylene units of the polymer repeat unit that are susceptible to substitution when oxidized or the polymer film requiring an increased break-in period (Kerszulis, J.A., et al., “Follow the Yellow Brick Road: Structural Optimization of Vibrant Yellow-to-Transmissive Electrochromic Conjugated Polymers”, Macromolecules, 47 (16): 5462-5469 (2014); Amb, C. M., et al., “Propylenedioxythiophene (ProDOT)-Phenylene Copolymers Allow a Yellow-to-Transmissive Electrochrome”, Polym. Chem., 2 (4): 812-814 (2011)). Regardless, this served as an area of improvement and motivates further design motifs to improve long-term redox stability.

### Example 13

A goal of this project was to demonstrate the utility of H₂DPP as a useful building block for simplifying the synthesis of conjugated polymers. The synthetic complexity (SC) was quantified using the equation developed by Po et al. as follows:

$$S C = 35 \frac{N S S}{N S {S}_{max}} + 25 \frac{log ( RY )}{log ( {RY}_{max} )} + 15 \frac{NUO}{{NUO}_{max}} + 15 \frac{N C C}{N C {C}_{max}} + 10 \frac{N H C}{N H {C}_{max}}$$

SC provided a reasonable starting point for comparing the synthetic complexity of H₂DPP-co-ProDOT to the field. In the above equation, the 5 variables were defined as the number of synthetic steps (NSS), the reciprocal yield of monomers (RY), the number of operations required for purification of monomers (NUO), the number of column chromatography purifications (NCC), and the number of hazardous materials used (NHC), all of which were assigned a weighted value based on the influence each step has on potential cost implications, such as personnel or waste disposal.

### Results

As shown in entry 5 of Table 2 below, the SC of H₂DPP-co-ProDOT was calculated to be 13.8 and this represented a synthetically simple conjugated polymer.

TABLE 3
Synthetic complexity analysis of selected
conjugated polymers and H₂DPP-co-ProDOT.^{a}

| Entry | Polymer | NSS | RY | NUO | NCC | NHC | SC |
|---|---|---|---|---|---|---|---|
| 1 | P3HT | 3 | 1.1 | 4 | 0 | 4 | 7.8 |
| 2 | PTQ10 | 3 | 1.1 | 5 | 1 | 6 | 9.7 |
| 3 | Cost-Effective PTQ10 | 5 | 2.1 | 8 | 1 | 9 | 18.4 |
| 4 | poly(ProDOT) | 5 | 3.1 | 1 | 4 | 9 | 21.3 |
| 5 | H₂DPP-co-ProDOT | 3 | 3.1 | 0 | 1 | 7 | 13.8 |

^{a}For a comprehensive tabulation of synthetic complexity for conjugated polymers, readers are directed to the work described in Ref. 5.

Poly(3-hexylthiophene) (P3HT) (entry 1, Table 2) had a synthetic complexity value of 7.8 but was hampered by using Grignard reagents and, ultimately, possessed modest device performance metrics (Baran, D., et al., “Reducing the Efficiency-Stability-Cost Gap of Organic Photovoltaics with Highly Efficient and Stable Small Molecule Acceptor Ternary Solar Cells”, Nat. Mater., 16 (3): 363-369 (2017); Guo, X. et al., “High Efficiency Polymer Solar Cells Based on Poly(3-Hexylthiophene)/Indene-C70 Bisadduct with Solvent Additive”, Energy Environ. Sci., 5 (7): 7943-7949 (2012)). The synthetic complexity of PTQ10 (entry 2, Table 2) also was calculated to be synthetically simple, as expected, due to the minimization of preparatory steps. However, the synthesis of PTQ10 monomers involves the use of potassium tert-butoxide, which is a flammable solid that is classified as self-heating in large quantities. Additionally, starting material and precursors are expensive, and polymers are synthesized via Stille cross-coupling polymerizations, which generate stoichiometric amounts of trialkyltin waste. Rech et al. performed a cost analysis of PTQ10 and suggest current synthetic approaches are cost prohibitive. These drawbacks limit the scalability and commercial manufacturing of this polymer and motivated the pursuit of alternative synthetic approaches that ultimately reduced the price per gram to about 1/7th the original cost (Rech, J. J., et al., “Designing Simple Conjugated Polymers for Scalable and Efficient Organic Solar Cells”, ChemSusChem, 14 (17): 3561-3568 (2021)). Notably, when calculating the synthetic complexity of the proposed cost-effective route for PTQ10 reported by Rech et al. (entry 3, Table 2), the addition of synthetic steps led to nearly doubling the synthetic complexity compared to the original synthetic strategy and resulted in a copolymer more synthetically complex than H₂DPP-co-ProDOT. While both PTQ10 approaches represented simple synthetic strategies for conjugated polymers, Stille cross-coupling polymerizations were used in both syntheses, which is detrimental to scalability efforts due to the generation of stoichiometric amounts of toxic waste.

Finally, given the potential applicability in redox applications, the SC of H₂DPP-co-ProDOT was compared to poly(ProDOT) (SC=21.3, entry 4, Table 2) and H₂DPP-co-ProDOT was calculated to be less synthetically complex than poly(ProDOT)). H₂DPP has been shown to be a viable monomer to simplify the synthesis of conjugated polymers for, potentially, both solid-state and redox applications. H₂DPP-co-ProDOT was synthetically simple and was able to remove toxic reagents (tin) and air/moisture sensitive reagents (Grignards), showing H₂DPP-containing copolymers offer significant advantages compared to other conjugated systems.

### Example 14

### Methods

All chemicals were purchased from commercial sources and used as received unless otherwise noted. Anhydrous N,N-dimethylacetamide (DMAc) was degassed with argon (Ar) for 15 minutes before use. 60 Å silica gel (200-400 mesh) was used for column chromatography.

¹H and ¹³C NMR spectra were collected on a Bruker Ascend 400 MHz NMR spectrometer with a 10-15 mg/mL nominal sample concentration in CDCl₃. Peaks were referenced to the residual CDCl₃ peak (¹H: δ=7.26 ppm; ¹³C: δ=77.23 ppm).

Solution absorbance spectra were acquired using a Varian Cary 4000 dual beam UV-vis-near-IR spectrophotometer scanning from 300 to 800 nm using a 20-40 μg/mL concentration in toluene.

Solution emission spectra were acquired using an Ollis is DM45 spectrofluorimeter scanning from 10 nm above λₘₐₓ^{abs} to 800 nm using a toluene solution with nominal concentration of 20-40 μg/mL.

Solution quantum yield was acquired using a Horiba Scientific Fluorolog-QM 75-11 equipped with an integrating K-sphere.

Thermogravimetric analysis (TGA) measurements were made using a PerkinElmer TGA 8000 using a temperature range of 30° C. to 900° C. with a heating rate of 5° C./min. The thermal degradation temperature (T_{d}) was obtained at 5% mass loss of a 5-10 mg polymer sample in a ceramic pan.

Differential scanning calorimetry (DSC) measurements were obtained using a Shimadzu DSC-60 with a heat-cool-heat cycle from 30° C. to 310° C. using a heating rate of 10° C./min.

Spectroelectrochemistry measurements were performed using a Varian Cary 5000 Scan dual-beam UV-vis-near-IR spectrophotometer.

The absorbances collected with this same spectrophotometer were converted to colorimetric coordinates using Star-Tek colorimetry software using a D50 illuminant, 2 deg observer, and the CIELAB L*a*b* color space.

Electrochemical measurements were performed using an EG&G Princeton Applied Research model 273A potentiostat/galvanostat under CorrWare control in a three-electrode cell configuration, using ITO/glass (Delta Technologies Inc., 7×50×0.7 mm, sheet resistance, Rₛ 8-12 Ω/sq) as the working electrode, an Ag/AgCl reference electrode (calibrated versus the Fe/Fe⁺ redox couple, E_{1/2}=40 mV), and a Pt flag as the counter electrode, with a 50 mV/s scan rate. The ITO slides were cleaned by sequential sonication in acetone, acetonitrile, and isopropanol, followed by a 5 min phosphonic acid treatment (10.0 mM hexadecylphosphonic acid in ethanol). An electrolyte solution of 0.5 M tetrabutylammonium hexafluorophosphate (TBAPF₆, 98%, purified via recrystallization from hot ethanol) in propylene carbonate (PC) was used in all electrochemical and spectroelectrochemical measurements. PC was purified using a Vacuum Atmospheres solvent purifier.

Polymer films were spray-cast onto the ITO-coated glass slides using an Iwata airbrush at 20 psi from 2 mg/mL toluene solutions. Photography was performed in a light booth designed to exclude outside light with a D50 (5000K) lamp located at the back of the booth providing illumination, while using a Nikon D90 SLR camera with a Nikon 18-105 mm VR lens. Pictures are presented without manipulation except for cropping.

### Example 15

### Methods

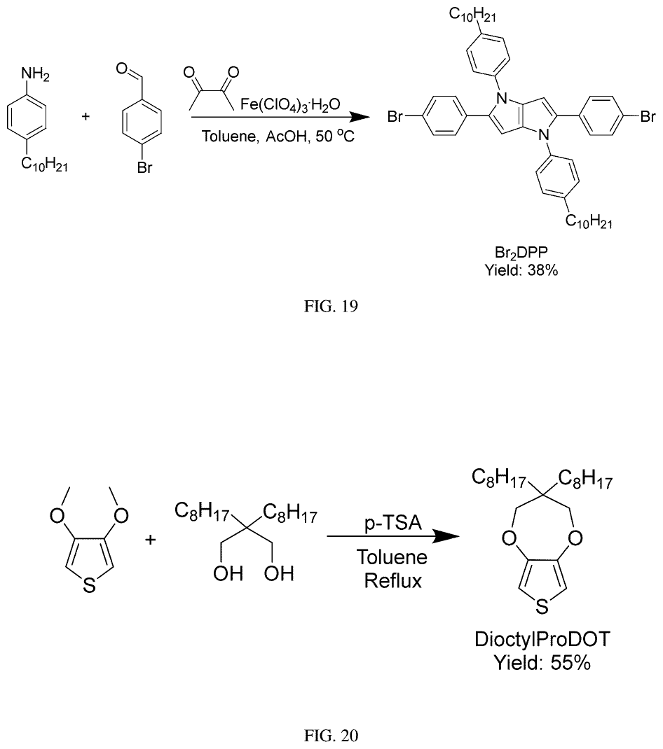

Br₂DPP was synthesized following a literature procedure report by Tasior et al. (Tasior, M., et al., “Method for the Large-Scale Synthesis of Multifunctional 1,4-Dihydro-Pyrrolo[3,2-b]Pyrroles”, J. Org. Chem., 85(21): 13529-13543 (2020)) with slight modifications, as shown in FIG. 19. 4-n-Decylaniline (1.86 g, 8.00 mmol), 4-bromobenzaldehyde (1.48 g, 8.00 mmol), toluene (6 mL), and glacial acetic acid (6 mL) were added to a 25 mL round-bottom flask equipped with a magnetic stir bar. The mixture was then heated and stirred for 1 h in an oil bath set to 50° C. After one hour of heating, Fe(ClO₄)₃·xH₂O (0.085 g) was added, followed by 2,3-butanedione (0.35 mL, 4.00 mmol). The final mixture was set to stir at 50° C. overnight with the flask open to air. After approximately 16 h, the reaction was removed from the oil bath and allowed cool to room temperature. The reaction precipitate was collected via vacuum filtration and washed with chilled methanol, followed by washing with chilled acetone until only a pale-yellow powder remained on the filter paper. The precipitate was the anticipated product and was dried under vacuum overnight. This assumption was confirmed after structural analysis, and the dihalogenated H₂DPP was isolated in respectable yields.

### Results

NMR data is displayed in FIGS. 6-7, and yields are as follows Yield: 1.22 g (1.52 mmol, 38%). ¹H NMR (400 MHz, CDCl₃, 25° C.), δ: 0.96 (t, 6H), 1.30-1.35 (m, 28H), 1.66 (m, 4H), 2.65 (m, 4H), 6.39 (s, 2H), 7.08 (d, 4H), 7.19-7.20 (m, 8H), 7.33 (d, 4H). ¹³C NMR: (400MHz, CDCl₃, 25° C.), δ: 14.11, 22.69, 29.34, 29.50, 29.62, 31.33, 31.92, 35.49, 94.62, 120.10, 129.15, 129.47, 131.24, 131.99, 132.63, 134.83, 137.31, 140.85 Anal. calc'd for C₅₀H₆₀Br₂N₂: C 70.75, H 7.12, Br 18.83, N 3.30 Found: C 70.95, H 7.03, N 3.33.

### Example 16

### Methods

The scalable preparation of Br₂DPP was achieved using the procedure described above but implementing a 10x increase in molar equivalents. For the initial mixture, 80.0 mmol of 4-n-decylaniline and 4-bromobenzaldehyde, 60 mL of toluene, and 60 mL of glacial acetic acid were combined into a 250 mL round-bottom flask. The final mixture included the addition of Fe(ClO₄)₃·xH₂O (0.850 g) and 2,3-butanedione (3.5 mL, 40.0 mmol). Purification was performed using the same protocol as described in Example 12.

### Results

The yield was 14.5 g (19.6 mmol, 49%). Analytical characterization and purity matched data from Example 12.

### Example 17

### Methods

A representative reaction scheme is shown in FIG. 20. First, 3,4-dimethoxythiophene (1.44 g, 10.0 mmol), 2,2-di-n-octyl-1,3-propanediol (4.89 g, 20.0 mmol), p-toluenesulfonic acid (0.172 g, 1.00 mmol), and 100 mL of toluene were combined in a 250 mL flask equipped with a Soxhlet extractor with 4 Å molecular sieves in a cellulose thimble. The reaction mixture was placed in an oil bath and refluxed overnight. After the allotted reaction time, the reaction mixture was cooled to room temperature, then poured into water (about 100 mL), and extracted with DCM (about 60 mL). The extract was washed with water (about 100 mL) before being dried with Na₂SO₄, filtered, and concentrated via rotary evaporation. The crude product was purified via column chromatography on silica gel using 3:2 hexanes:DCM as the mobile phase. The product was then dried overnight under vacuum yielding a clear oil.

### Results

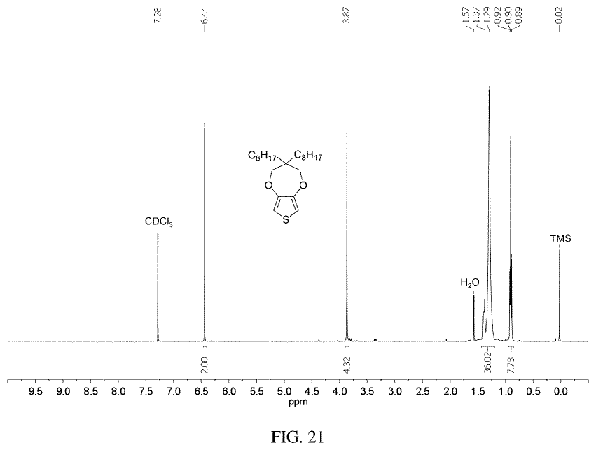

Analytical characterization was consistent with previously published reports. NMR data is displayed in FIG. 21, and yields are as follows Yield: 2.09 g (5.50 mmol, 55%). ¹H NMR (400 MHz, CDCl3), δ=6.39 (s, 2H), 3.90 (s, 4H), 1.44-1.19 (m, 20H), 0.93-0.80 (m, 6H)

### Example 18

### Methods

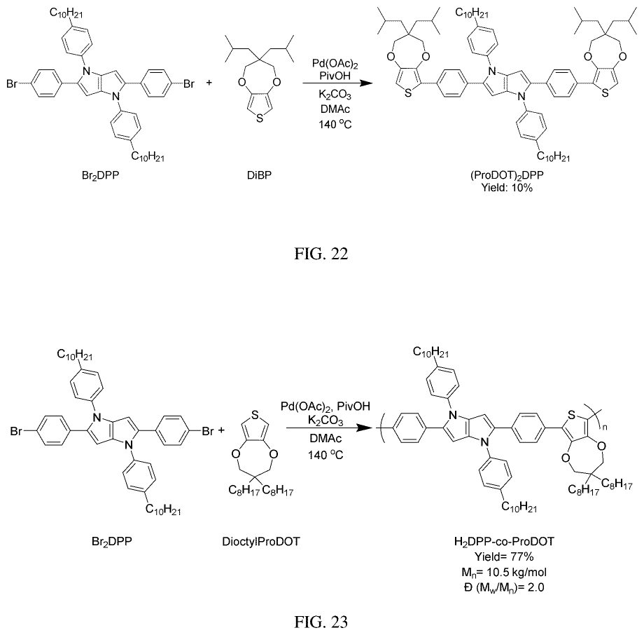

A representative reaction scheme is shown in FIG. 22. Br₂DPP (100 mg, 0.118 mmol), diisobutylProDOT (DiBP) (380 mg, 1.42 mmol), Pd(OAc)₂ (2.1 mg, 0.009 mmol), pivalic acid (14.4 mg, 0.142 mmol), and K₂CO₃ (24.4 mg, 0.148 mmol) were added to a 10 mL round-bottom flask equipped with a magnetic stir bar. The flask was sealed with a rubber septum and rendered inert via 3 vacuum/refill cycles with Ar. DMAc (0.60 mL) was added to the flask via syringe, and the flask was placed in an oil bath heated to 140° C. The reaction was monitored via thin-layer chromatography (TLC) by taking aliquots every 5 minutes. After about 60 min, all the H₂DPP starting material was consumed, and the reaction was then quenched with ethyl acetate. The reaction mixture was cooled to room temperature, then poured into water (about 100 mL), and extracted with DCM (about 60 mL). The extract was washed with water (about 50 mL) and brine (about 50 mL). The organic phase was dried over Na₂SO₄, filtered, and concentrated via rotary evaporation. The crude product was purified by column chromatography using 4:1 hexane:DCM as the eluent. The sample was dried overnight under a vacuum yielding a yellow solid.

### Results

Yields and NMR data are as follow. Yield: 13.7 mg (0.012 mmol, 10%). ¹H NMR (400 MHz, CDCl3), δ=7.56 (d, 4H), 7.18-7.27 (m, 12H), 6.43 (s, 2H), 6.37 (s, 2H), 3.89-4.07 (m, 8H), 2.65 (t, 4H), 1.81 (m, 8H), 1.28 (br, m), 1.00 (br, d).

### Example 19

### Methods

A representative reaction scheme is shown in FIG. 22. Br₂DPP (200 mg, 0.236 mmol), Pd(OAc)₂ (1.1 mg, 0.005 mmol), PivOH (7.2 mg, 0.071 mmol), and K₂CO₃ (81.5 mg, 0.59 mmol) were added to a Schlenk flask equipped with a magnetic stir bar and sealed with a rubber septum. The flask was rendered inert via 3 vacuum/refill cycles with Ar. DioctylProDOT (89.8 mg, 0.236 mmol) was weighed in a vial and transferred to the reaction flask using degassed DMAc. The reaction was lowered into an oil bath set to 140° C. and stirred overnight (16-20 h). After the allotted time, the reaction flask was removed from the oil bath and cooled to room temperature. The reaction mixture was precipitated in an excessive amount of methanol (MeOH) and stirred for 30 minutes. The precipitate was then collected in a Soxhlet thimble. The polymer was purified with subsequent washes with MeOH and acetone before being extracted with chloroform. The extracted solution was concentrated via rotary evaporation and then reprecipitated into MeOH. The precipitate was then filtered via vacuum filtration, washed with MeOH, and dried under a vacuum overnight.

### Results

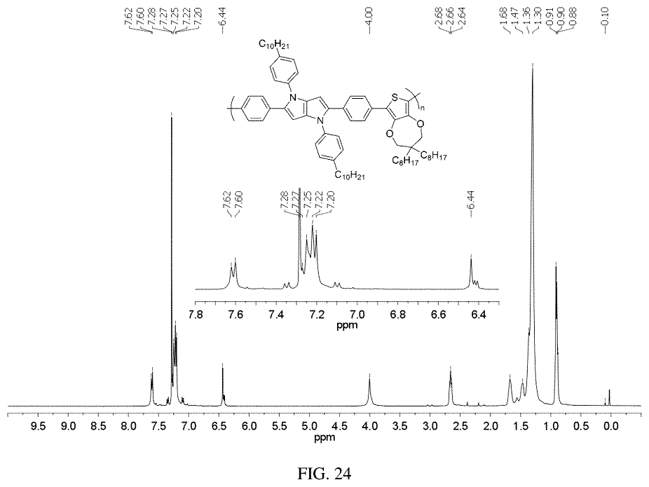

NMR data is displayed in FIG. 24, and yields are as follows. Yield: 195 mg (77%). Mₙ=10.5 kg/mol, Ð=2.0, ¹H NMR (400 MHz, CDCl3), δ=1.21-1.41 (br, m), 1.43-1.52 (br, m), 1.63-1.73 (br, m), 2.62-2.70 (br, t), 6.35-6.47 (br, s), 7.18-7.30 (br, m), 7.58-7.64 (br, d). Anal. calc'd for C₇₃H₉₈N₂O₂S: C 81.97, H 9.42, N 2.78, 0 2.99, S 3.00 Found: C 80.99, H 9.09, N 2.78, S 2.73.

That which is claimed is:

1. A composition comprising a copolymer comprising a monomer derived from a pyrrole.

2. The composition of claim 1, wherein the monomer is derived from a pyrrolopyrrole.

3. The composition of claim 2, wherein the pyrrolopyrrole is pyrrolo[3,2-b]pyrrole (H₂DPP).

4. The composition of claim 1 further comprising a monomer derived from a dioxythiophene.

5. The composition of claim 4, wherein the dioxythiophene is 3,4-propylenedioxythiophene (ProDOT).

6. The composition of claim 1 further comprising a monomer of thienopyrroledione (TPD) or diketopyrrolopyrrole (ketoDPP).

7. The composition of claim 1 comprising a copolymer comprising a repeating unit, wherein the repeating unit is of the following formula 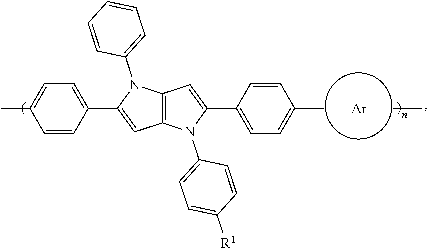
wherein R¹ comprises a functional group selected from the following formulas or C₁₀H₂₁ 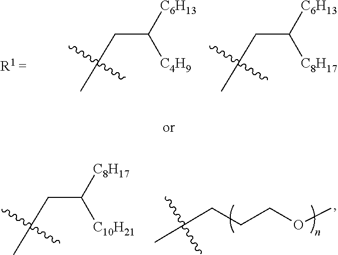
wherein Ar is an aromatic compound selected from the following formulas 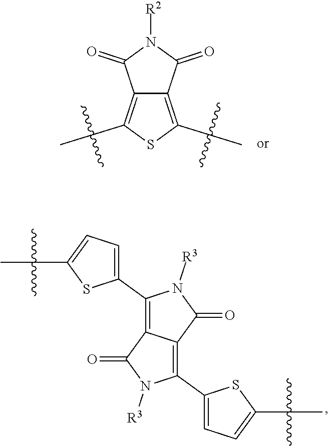
and wherein R² is C₈H₁₇ and R³ is EtHx.

8. A method of synthesizing a copolymer repeat unit derived from a pyrrole and a monomer derived from a dioxythiophene, the method comprising:
a. synthesizing a dihalogenated, pyrrole-derived monomeric unit;
b. synthesizing a dioxythiophene-derived monomeric unit;
c. synthesizing the copolymer through direct heteroarylation polymerization (DHAP) of the dibrominated, pyrrole-derived monomeric unit and the dioxythiophene-derived monomeric unit.

9. The method of claim 8, wherein the pyrrole-derived monomeric unit is dibrominated.

10. A method of incorporating at least one H₂DPP molecule into a repeat unit of a copolymer, the method comprising a reaction comprising:
a. providing a dibrominated, pyrrole-derived monomeric unit;
b. providing an aromatic compound; and
c. contacting the dibrominated, pyrrole-derived monomeric unit with the aromatic compound in a solution.

11. The method of claim 10, wherein the dibrominated, pyrrole-derived monomeric unit is of the following formula 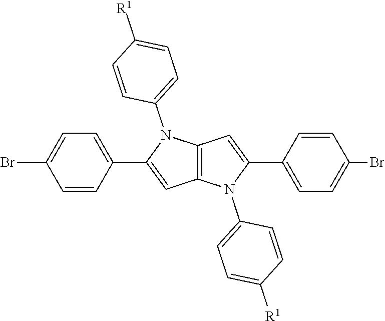
and wherein R¹ comprises a functional group selected from the following formulas or C₁₀H₂₁

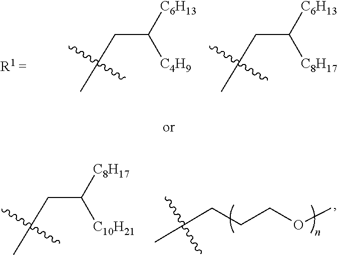

12. The method of claim 10, wherein the aromatic compound is selected from the following formulas 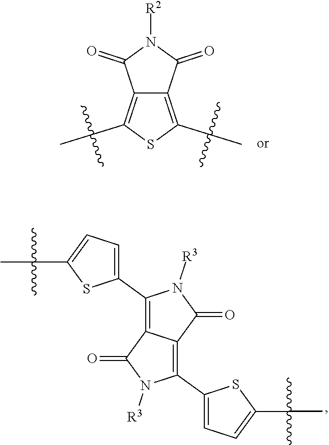
and wherein R² is C₈H₁₇ and R³ is EtHx.

13. The method of claim 10, wherein the solution comprises Pd(OAc)₂ and PivOH.

14. The method of claim 13, wherein the solution further comprises PCy₃, HBF₄, and CS₂CO₃.

15. The method of claim 14, wherein the solution further comprises DMAc.

16. The method of claim 10, wherein the reaction is carried out at a temperature from about 50° C. up to about 140° C.

17. The method of claim 10, wherein the reaction is carried out at a temperature of about 140° C.
